SEQUENCE LISTING (24 sequences)

SEQ ID NO: 1 - DNA, 24 bp, Artificial Sequence
  GuaB2 primer
cgttcaggcgcaacaggccgttgt

SEQ ID NO: 2 - DNA, 22 bp, Artificial Sequence
  GuaB3 primer
ggctgcgattggcgaggtagta

SEQ ID NO: 3 - DNA, 35 bp, Artificial Sequence
  GuaB4 primer
ggtgatcccgggcgtcaaacgtcagggcttcttta

SEQ ID NO: 4 - DNA, 35 bp, Artificial Sequence
  GuaB5 primer
ttgacgcccgggatcaccaaagagtccccgaacta

SEQ ID NO: 5 - DNA, 20 bp, Artificial Sequence
  GuaB6 primer
gcaacaactcctgctggtta

SEQ ID NO: 6 - DNA, 20 bp, Artificial Sequence
  GuaB7 primer
agaccgaggatcactttatc

SEQ ID NO: 7 - DNA, 19 bp, Artificial Sequence
  GuaB10 primer
aggaagtttgagaggataa

SEQ ID NO: 8 - DNA, 20 bp, Artificial Sequence
  P1 primer
gtgtaggctggagctgcttc

SEQ ID NO: 9 - DNA, 20 bp, Artificial Sequence
  P2 primer
catatgaatatcctccttag

SEQ ID NO: 10 - DNA, 72 bp, Artificial Sequence
  FliCP1 primer
atggcacaagtcattaatacaaacagcctgtcgctggttgacccagaataatgtgtaggctggagctgcttc

SEQ ID NO: 11 - DNA, 74 bp, Artificial Sequence
  FliCP2 primer
cgcattaacgcagtaaagagaggacgttttgcggaacctggttmgcctgcgccacatatgaatatcctccttag

SEQ ID NO: 12 - DNA, 26 bp, Artificial Sequence
  FliC1 primer
atggcacaagtcattaatacaaacag

SEQ ID NO: 13 - DNA, 25 bp, Artificial Sequence
  FliC2 primer
cgcattaacgcagtaaagagaggac

SEQ ID NO: 14 - DNA, 20 bp, Artificial Sequence
  FliC3 primer
tatcggcaatctggaggcaa

SEQ ID NO: 15 - DNA, 72 bp, Artificial Sequence
  FljBAP1 primer
atggcacaagtaatcaacactaacagtctgtcgctgctgacccagaataactgtgtaggctggagctgcttc

SEQ ID NO: 16 - DNA, 71 bp, Aartificial Sequence
  FljBAP2 primer
ttattcagcgtagtccgaagacgtgatcctgctcacccagtcaaacataaccatatgaatatcctccttag

SEQ ID NO: 17 - DNA, 23 bp, Artificial Sequence
  FljBA5 primer
cagcgtagtccgaagacgtgatc

SEQ ID NO: 18 - DNA, 22 bp, Artificial Sequence
  FljBA6 primer
acactaacagtctgtcgctgct

SEQ ID NO: 19 - DNA, 2903 bp, Artificial Sequence
  Salmonella Enteritidis
ctgatcgaacaagccttcggcctgcagtttggcttttagctgctcatatttctgctgcaacaagccttcgcccgcgggctgcatactttcggcgatgatttgataatcgccgcgcggctcgtacagcgtaatgttggcgcgtaccagcacctgctgcccgtgctgcgggcggaacgtcacccggcgattgctgttacggaacatcgcacagcgcacctgagcggtatcgtctttgagcgtaaagtaccagtggcccgacgcaggctgcgtgaaattagaaatctcgccgctgatccatacctgtcccatctcctgttctaacagcagacgaaccgtctggttaaggcggcttacggtaaaaattgaggaagtttgagaggataacatgtgagcgggatcaaattctaaatcagcaggttattcaatcgatagtaacctgctcacgggggatcgcaagcactatttgcaaaaaaatgtagatgcaaccgattacgttctgtataatgccgcgggaatatttattaacctcccaggcgagatattgcccatgctacgtatcgctaaagaagctctgacgtttgacgacgtcctccttgttcccgctcactccaccgttttgccgaatactgccgatctcagcacgcagttgacgaaaactattcgtctgaatattcctatgctttctgcggcgatggacaccgtgacggaagcgcgcctggcaattgccctggcccaggaaggcggcattggttttatccacaaaaacatgtctattgagcgccaggcggaagaagttcgccgcgtgaagaaacacgagtccggcgtagtgaccgacccgcagaccgtcctgccaaccaccacgttgcatgaagtgaaagccctgaccgagcgtaacggttttgcgggctatccggtggtgactgaagataacgagctggtgggtatcatcaccggtcgtgacgtgcgttttgtgactgacctgaaccagccggtgagtgtctacatgacaccgaaagagcgtctggtgaccgttcgtgaaggcgaagcccgtgaagtcgtgctggcaaaaatgcacgaaaaacgcgtagaaaaagcgctggtcgttgatgataacttccatctgcttggcatgattaccgtaaaagatttccagaaagcggaacgtaaaccaaactcctgtaaagatgagcagggccgtttacgtgtcggcgcggcggtcggcgcaggcgcgggcaacgaagagcgcgttgacgcgctggtggcggcaggcgttgacgtactgctgatcgactcctctcacggtcactctgaaggcgtgttgcaacgtatccgtgagacgcgtgctaaatatcctgacctgcaaatcatcggcgggaacgttgcgacgggcgcaggcgctcgcgcactggcggaagccggttgcaacgcggtgaaagtgggtatcggcccgggttccatctgtacgactcgtatcgggactggtgtgggcgttccgcagatcaccgctgtttctgacgcggtggaagcgctggaaggcaccgggattccggttatcgctgacggcggtatccgtttctccgnnnacatcgccaaagccatcgccgcaggcgcgagcgcggtaatggtgggttctatgctggccggtaccgaagaatcnccgggcgaaatcgaactctaccagggccgttcttacaaatcttatcgcggtatgggttctctgggcgcgatgtccaaaggttcctccgaccgttacttccagagcgacaacgccgccgacaaactggtgccggaaggtatcgaaggccgcgtagcctataaaggtcgcctgaaagagatcattcaccagcagatgggcggcctgcgctcctgtatggggctgaccggttgtgctaccatcgacgaactgcgtactaaagcggagtttgtgcgtatcagcggtgcgggtatccaggaaagccacgttcacgacgtgaccatcaccaaagagtccccgaactaccgtctgggctcctgattttcttcgcccgaccttcgcgtcgggcgatttatttaatctgtttcacttgcctcggaataagcgtcaatgacggaaaacattcataagcatcgcatcctcattctggacttcggttctcagtacactcaactggttgcgcgccgcgtgcgtgagctgggtgtttactgtgaactgtgggcgtgggatgtgacagaagcacaaattcgtgacttcaacccaagcggcattattctttccggcggcccggaaagcaccaccgaagaaaacagcccgcgcgcgccgcagtatgtctttgaagcaggcgtgccggtatttggcgtttgctatggtatgcagaccatggcgatgcagcttggcggtcatgtagaaggttctaatgagcgtgaatttggttatgcgcaggtcgaagtgttgaccgacagcgcgctggttcgcggtattgaagattccctgaccgcagacggcaaaccgctgctggacgtgtggatgagccacggcgataaagtgacggcgattccgtccgacttcgtgaccgtcgccagcaccgagagctgcccgttcgccattatggccaacgaagaaaaacgcttctacggcgtacagttccacccggaagtgactcacacccgccagggtatgcgcatgctggagcgttttgtacgcgatatctgccagtgtgaagcgttgtggacgccggcgaagatcatcgatgacgccgtggcgcgcattcgcgagcaggtaggcgacgataaagtgatcctcggtctctccggcggcgtggattcttccgtcaccgccatgctgctgcaccgcgcgatcggtaaaaatctgacctgtgttttcgtcgacaacggcctgttgcgcctgaacgaagccgagcaggtga

SEQ ID NO: 20 - DNA, 1995 bp, Artificial Sequence
  Sequence of the delta-guaB fragment of S. Enteritidis cloned in pUC18
ggctgcgattggcgaggtagtagtccatcgccatgcccagacgctgctggcgganctggatctgacgcaacaactcctgctggttacggctgacaatttctgccgctgctgatggcgtagcgcgcgcaggtcggcgacaaaatcagctatcgtgacgtccgtttcgtgaccgacggcgctgaccaccggaatgcggctggcaaaaatcgcccgcgccacgcgttcgtcgttaaaactccacaaatcttccagcgaaccgccgccgcgcccgacgatcagtacatcacattcgccgcgcgcgttcgccagttcgatagcacgaacgatctgccccggcgcgtcgtcgccctggactgcggttggatagataataacgggcagggatgggtcacgacgctttagcacgtggagaatatcgtgcagcgccgcgccggttttcgaagtgatcaccccaacgcagtgggccggggagggcaacggctgtttatgctgctgatcgaacaagccttcggcctgcagtttggcttttagctgctcatatttctgctgcaacaagccttcgcccgcgggctgcatactttcggcgatgatttgataatcgccgcgcggctcgtacagcgtaatgttggcgcgtaccagcacctgctgcccgtgctgcgggcggaacgtcacccggcgattgctgttacggaacatcgcacagcgcacctgagcggtatcgtctttgagcgtaaagtaccagtggcccgacgcaggctgcgtgaaattagaaatctcgccgctgatccatacctgtcccatctcctgttctaacagcagacgaaccgtctggttaaggcggcttacggtaaaaattgaggaagtttgagaggataacatgtgagcgggatcaaattctaaatcagcaggttattcaatcgatagtaacctgctcacgggggatcgcaagcactatttgcaaaaaaatgtagatgcaaccgattacgttctgtataatgccgcggcaatatttattaacctcccaggtgagatattgcccatgctacgtatcgctaaagaagctctgacgtttgacgcccgggatcaccaaagagtccccgaactaccgtctgggctcctgattttcttcgcccgaccttcgcgtcgggcgatttatttaatctgtttcacttgcctcggaataagcgtcaatgacggaaaacattcataagcatcgcatcctcattctggacttcggttctcagtacactcaactggttgcgcgccgcgtgcgtgagctgggtgtttactgtgaactgtgggcgtgggatgtgacagaagcacaaattcgtgacttcaacccaagcggcattattctttccggcggcccggaaagcaccaccgaagaaaacagcccgcgcgcgccgcagtatgtctttgaagcaggcgtgccggtatttggcgtttgctatggtatgcagaccatggcgatgcagcttggcggtcatgtagaaggttctaatgagcgtgaatttggttatgcgcaggtcgaagtgttgaccgacagcgcgctggttcgcggtattgaagattccctgaccgcagacggcaaaccgctgctggacgtgtggatgagccacggcgataaagtgacggcgattccgtccgacttcgtgaccgtcgccagcaccgagagctgcccgttcgccattatggccaacgaagaaaaacgcttctacggcgtacagttccacccggaagtgactcacacccgccagggtatgcgcatgctggagcgttttgtacgcgatatctgccagtgtgaagcgttgtggacgccggcgaagatcatcgatgacgccgtggcgcgcattcgcgagcaggtaggcgacgataaagtgatcctcggtctctccggcggcgtggattcttccgtcaccgccatgctgctgcaccgcgcgatcggtaaaaatctgacctgtgttttcgtcgacaacggcctgttgcgcctgaacg

SEQ ID NO: 21 - DNA, 349 bp, Artificial Sequence
  Nucleotide sequence of the S. Enteritidis PCR fragment, which includes the guaB deletion, obtained using primer GuaB10
catgtgtgagcgggatcaaattctaaatcagcaggttattcaatcgatagtaacctgctcacgggggatcgcaagcactatttgcaaaaaaaatgtagatgcaaccgattacgttctgtataatgcccggcaatatttattaacctcccaggtgagatattcgccatgtacgtatcgctaaagaagccctgacgtttgacgcccgtgtaggctggagctgcttcgaagttcctatactttctagagaataggaactccggaataggaactaaggaggatattcatatgggatcaccaaagatccccgaactaccgtctgggctctgattttcttcgcccgaccttcgcg

SEQ ID NO: 22 - DNA, 1553 bp, Artificial Sequence
  Salmonella Typhimurium
atttgcaaaaaaatgtagatgcaaccgattacgttctgtataatgccgcggcaatatttattaacctcccaggtgagatattgcccatgctacgtatcgctaaagaagccctgacgtttgacgacgtcctccttgttcccgctcactccaccgttttgccgaatactgccgatctcagcacgcagttgacgaaaactattcgtctgaatattcctatgctttctgcggcgatggacaccgtgacggaagcgcgcctggcaattgccctggcccaggaaggcggcattggttttatccacaaaaacatgtccattgagcgccaggcggaagaagttcgccgcgtgaagaaacacgagtccggcgtagtgaccgacccgcagaccgtcctgccaaccaccacgttgcatgaagtgaaagccctgaccgagcgtaacggttttgcgggctatccggtggtgactgaagataacgagctggtgggtatcatcaccggtcgtgacgtgcgttttgtgactgacctgaaccagccggttagcgtttacatgacgccgaaagagcgtctggtgaccgttcgtgaaggcgaagcccgtgaagtcgtgctggcaaaaatgcacgaaaaacgcgtagaaaaagcgctggtcgttgatgataacttccatctgcttggcatgattaccgtaaaagatttccagaaagcggaacgtaaaccaaactcctgcaaagatgagcagggccgtttacgtgtcggcgcggcggtcggcgcaggcgcgggcaacgaagagcgcgttgacgcgctggtggcggcaggcgttgacgtactgctgatcgactcctctcacggtcattcagaaggcgtgttgcaacgtatccgtgaaacccgtgctaaatatcctgacctgcaaatcatcggcggcaacgtcgcgacaggcgcaggcgctcgcgcactggcggaagccggttgcagcgcggtgaaagtcggtattggcccgggttccatctgtaccactcgtatcgtgactggcgtgggcgttccgcagatcaccgctgtttctgacgcagttgaagcgctggaaggtaccggtattccggttatcgctgacggcggtatccgtttctccggcgacatagccaaagcgattgccgcaggtgcaagcgcggtaatggtgggttccatgctggcgggtacggaagaatccccgggcgaaatcgaactctaccagggccgttcttacaaatcttaccgcggcatgggctcgctgggtgcgatgtccaaaggttcctccgaccgttacttccagagcgacaacgccgctgacaaactggtgccggaaggtatcgaaggtcgcgtagcctataaaggtcgcctgaaagagatcattcaccagcagatgggcggcctgcgctcctgtatggggctgaccggttgtgctaccatcgacgaactgcgtactaaagcggagtttgtgcgtatcagcggtgcgggcattcaggaaagccacgttcacgacgtgaccatcaccaaagagtccccgaactaccgtctgggctcctga

SEQ ID NO: 23 - DNA, 193 bp, Artificial Sequence
  Nucleotide sequence obtained after sequencing the PCR fragment amplified with primers FliC1-FliC2 on total DNA of the mutants SM73 and SM89, using primers FliC1 and FliC2
ctatggcacaagtcattaatacaaacagcctgtcgctggttgacccagaataatgtgtaggctggagctgcttcgaagttcctatactttctagagaataggaacttcggaataggaactaaggaggatattcatatgtggcgcaggcgaaccaggttccgcaaaacgtcctctctttactggcgttaatgcg

SEQ ID NO: 24 - DNA, 140 bp, Artificial Sequence
  Nucleotide sequence obtained after sequencing the PCR fragment amplified with primers FljBA6-FljBA5 on total DNA of the S. Typhimurium mutant SM48, using primer FljBA6
agaataactgtgtaggctggagctgcttcgaagttcctatactttctagagaataggaacttcggaataggaactaaggaggatattcatatggttatgtttgactgggtgagcaggatcacgtcttcggactacgctgt

(19) United States
(12) Patent Application Publication
(10) Pub. No.: US 2011/0052635 A1
(43) Pub. Date: Mar. 3, 2011

(54) LIVE ATTENUATED SALMONELLA VACCINE

(75) Inventors: Henri Marcel Jozef De Greve, Sint-gillis (BE); Connie Theresia Adriaensen, Ghent (BE); Jean-Pierre Ernest Clement Hernalsteens, Sint-Pieters-Woluwe (BE)

(73) Assignee: Vrije Universiteit Brussel, Brussels (BE)

(21) Appl. No.: 12/293,786

(22) PCT Filed: Mar. 20, 2006
(86) PCT No.: PCT/BE06/00020

§ 371 Date: Sep. 24, 2009

(51) Int. Cl. A61K 39/02; C12N 1/21; A61P 31/04; C12Q 1/02

(52) U.S. Cl. 424/234.1; 435/252.3; 435/252.33; 435/29

(57) ABSTRACT

The present invention is related to double and triple attenuated mutant strains of a bacterium infecting veterinary species such as Salmonella enterica and/or (a pathogenic) Escherichia coli. The mutants of the invention contain at least one first genetic modification and at least one second genetic modification, said first modification in one or more motility genes, and said second modification in one or more genes involved in the survival or the proliferation of the pathogen in the host. The present invention further relates to live attenuated vaccines based on such mutants for preventing amongst others Salmonellosis and/or an infection by an E. coli pathogen in a veterinary species.

### FIELD OF THE INVENTION

The present invention relates to attenuated bacterial mutants, in particular attenuated Salmonella enterica mutants, and to a live attenuated vaccine comprising same. The double, triple, multiple mutants of the invention advantageously allow a serological distinction between vaccinated animals and (non-vaccinated) animals that have been exposed to a wild-type field such as wild-type field S. enterica.

### STATE OF THE ART

Salmonellae are Gram-negative, facultative anaerobic, motile, non-lactose fermenting rods belonging to the family Enterobacteriaceae. Salmonella are usually transmitted to humans by the consumption of contaminated foods and cause Salmonellosis. E. coli is another member of the family Enterobacteriaceae.

Salmonellae have been isolated from many animal species including, cows, chickens, turkeys, sheep, pigs, dogs, cats, horses, donkeys, seals, lizards and snakes.

95% of the important Salmonella pathogens belong to S. enterica, with S. enterica serovar Typhimurium (S. Typhimurium) and S. enterica serovar Enteritidis (S. Enteritidis) the most common forms.

Salmonella infections are a serious medical and veterinary problem world-wide and cause concern in the food industry. Contaminated food cannot be readily identified.

Control of Salmonellosis is important, to avoid potentially lethal human infections and considerable economic losses for the animal husbandry industry.

The ubiquitous presence of Salmonella in nature complicates the control of the disease just by detection and eradication of infected animals.

Several control strategies based on the principles of competitive exclusion and vaccination have been tested to control the infection of e.g. poultry.

Vaccination of farm animals is often considered as the most effective way to prevent zoonoses caused by e.g. Salmonella.

Whole-cell killed vaccines and subunit vaccines are used with variable results in the prevention of Salmonella infections in animals and in humans. Inactivated vaccines in general provide poor protection against Salmonellosis.

Live attenuated Salmonella vaccines are potentially superior to inactivated preparations owing to: (i) their ability to induce cell-mediated immunity in addition to antibody responses; (ii) oral delivery with no risk of needle contamination; (iii) effectiveness after single-dose administration; (iv) induction of immune responses at multiple mucosal sites; (v) low production cost; and (vi) their possible use as carriers for the delivery of recombinant antigens to the immune system.

The following attenuated mutant strains have been tested on their efficiency to induce a protective immune response in treated animals: (1) strains carrying mutations in the aro genes (Alderton et .al., 1991, Avian diseases 35:435-442; Schiemann and Montgomery, 1991, Veterinary Microbiology 27: 295-308), (2) strains carrying deletions in the cya (adenylate cyclase) and/or crp (cyclic AMP receptor) genes (U.S. Pat. No. 5,389,368; U.S. Pat. No. 5,855,879; U.S. Pat. No. 5,855,880; Hassan and Curtiss, 1997, Avian Diseases 41:783-791; Porter et al. 1993, Avian Diseases 37:265-273) and (3) strains that carry mutations in the genes of the guaBA operon of S. typhi (Wang et al., 2001, Infection and Immunity 69:4734-4741; WO99/58146 and U.S. Pat. No. 6,190,669).

So far only the vaccine strain Megan® Vacl, that carries deletions in the cya and crp genes, has been effective, at least in part. This strain does not provide full protection (http://www.meganhealth.com/meganvac.html).

McFarland and Stocker (1987, Microbial pathogenesis 3:129-141) reported on the virulence of guaA and guaB Tn10 insertion mutants of S. Typhimurium and S. dublin in BALE/c mice. At high dosage (2.5×10⁷ CFU), these authors reported a significant lethality of animals, resulting from the multiplication of the auxotrophic strain.

Also the ΔguaBA mutant of S. typhi proved no suitable candidate for sound and safe protection against typhoid fever. It showed a significant residual virulence in mice (Wang et al., 2001).

There is thus still a need for improved live attenuated Salmonella vaccine strains, as well as for improved live attenuated vaccine strains of bacteria infecting veterinary species in general.

Vaccinated animals often produce antibodies against different antigens of the pathogen. Problem is that vaccinated animals as such can no longer be distinguished from animals that have been in contact with a wild-type field strain such as a Salmonella field strain, and are possibly infected therewith.

There is thus also a need for improved live attenuated strains like live attenuated Salmonella vaccine strains that would make such distinction possible.

### AIMS OF THE INVENTION

An object of the present invention is to provide attenuated Salmonella enterica strains with a double or a triple mutation.

Another object of the present invention is to provide a live attenuated vaccine against Salmonellosis and methods of treatment based thereon.

Yet another object of the present invention is to provide attenuated Salmonella strains which are useful as live vector and as DNA-mediated vaccines expressing foreign antigens. Such strains are thus highly suitable for the development of vaccines including polyvalent vaccines.

Still another object of this invention is to provide a method to achieve S. enterica deletion mutants of the invention.

Still a further object of this invention is to provide an attenuated Salmonella strain that allows a serological distinction between vaccinated and non-vaccinated yet possibly infected animals.

Yet a further object of this invention is to provide the same materials and methods for the preparation of attenuated strains of a bacterium infecting veterinary species in general, more in particular poultry.

The general aim is to improve food safety and animal health.

### SUMMARY OF THE INVENTION

Some ΔguaB auxotrophic Salmonella enterica mutants with a deletion mutation in the guaB gene showed residual virulence. It was found that further modifications. (preferably deletions) in one or more genes involved in motility reduced the remaining virulence without affecting the immunogenic capacities of the strain.

A first aspect of the invention therefore relates to an attenuated mutant strain of a bacterium infecting veterinary species, in particular an attenuated S. enterica mutant strain, wherein said mutant strain contains at least one first genetic modification and at least one second genetic modification, said first modification in one or more (at least one) motility genes, and said second modification in one or more (at least one) genes involved in the survival or the proliferation of the bacterium or pathogen (e.g. S. enterica) in the host. The term “bacterium infecting veterinary species” in the context of the invention refers in particular to bacteria that are pathogenic to veterinary species, and which can be attenuated by the above genetic modifications. The bacterium infecting veterinary species may be a Gram-negative bacterium. Preferred are Gram-negative bacteria for poultry such as Salmonella, Pasteurella, Escherichia coli, etc. Most preferred are Salmonella enterica and (pathogenic) E. coli. By “pathogenic to” is meant that the bacterium, if not attenuated, is capable of causing an infectious disease in the veterinary species.

The genetic modifications of the invention advantageously lead to a null-function, in other words impair or affect the gene function. The modification in the present context is also referred to as an “impairing modification”. The modification is said to inactivate the gene in question. Advantageously, said inactivation results in attenuation, at least to a degree that the mutant strain is suitable for use in a live attenuated vaccine.

The genetic modification may be an insertion, a deletion, and/or a substitution of one or more nucleotides in said genes. Mutant strains according to the invention by such modification are affected in a motility gene function and in a gene function needed for the survival or the proliferation of the pathogen, leading to a null-function (no functional gene product formed) of the affected genes.

Deletion mutants are preferred, as an insertion mutant may revert, thereby restoring the pathogenicity of the strain.

The first modification is in one or more (1, 2, 3, . . . ) motility genes. Examples of a gene involved in motility are the genes encoding flagellin. The mutant of the invention may have a (impairing) modification in the fliC and/or the fljB or the fljBA genes respectively (fliC; fljB; fljBA; fliC and fljB; fliC and fljBA; . . . ). Advantageously mutants, in which all genes encoding flagellin are deleted, are incapable of swarming out on LB medium containing 0.4% agar and can thereby easily be distinguished from wild-type motile strains.

The second genetic modification is in one or more (1, 2, 3, . . . ) genes involved in the survival or the proliferation of the pathogen in its host. Such gene may be a house-keeping gene or a virulence gene. An example of a housekeeping gene that can lead to attenuated strains when the gene function is affected, is the guaB gene encoding the enzyme IMP dehydrogenase. Such mutant is incapable of forming de novo guanine nucleotides. Also possible are impairing modifications in the guaBA operon, advantageously leading to a null-function of the gene(s) encoding for or regulating proper IMP dehydrogenase activity.

Advantageously, the attenuated mutant strains of the invention are immunogenic.

The present invention in particular aims to provide attenuated S. Enteritidis and S. Typhimurium strains.

Preferably the genetic modifications of the invention are introduced into parent strain S. Enteritidis phage type 4 strain 76Sa88 or into parent strain S. Typhimurium 1491S96. The 76Sa88 strain is a clinical isolate from a turkey, obtained from the Veterinary and Agrochemical Research Centre, Groeselenberg 99, B-1180 Ukkel, Belgium, harboring the temperature sensitive replication plasmid pKD46, encoding the bacteriophage Lambda Red recombinase system. The 1491S96 strain is a clinical isolate from a chicken.

One of the attenuated S. enterica strains obtained according to the invention is S. Enteritidis strain SM73 having the deposit number deposit number LMG P-21642. Another example is the attenuated S. Typhimurium strain SM89 having the deposit number LMG P-21643.

A preferred mutant of the invention carries or comprises a genetic modification in the guaB gene and a genetic modification in the fliC gene.

Another preferred mutant of the invention carries or comprises a genetic modification in a guaB gene and a genetic modification in the fljBA genes.

Yet another preferred mutant of the invention carries or comprises a genetic modification in the guaB gene, a genetic modification in the fliC gene, and a genetic modification in the fljBA genes.

The attenuated strains of the invention are highly suitable for use in a live attenuated vaccine. The mutant strains of the invention may encode and express a foreign antigen.

Another aspect of the invention relates to a vaccine for immunizing a veterinary species against a bacterial infection, comprising:

a pharmaceutically effective or an immunizing amount of an attenuated mutant strain according to the invention; and

a pharmaceutically acceptable carrier or diluent. The present invention in particular relates to vaccines comprising attenuated mutant strains of S. enterica and/or E. coli.

In general about 10² cfu to about 10¹⁰ cfu, preferably about 10⁵ cfu to about 10¹⁰ cfu is administered (examples of a pharmaceutically effective or an immunizing amount). An immunizing dose varies according to the route of administration. Those skilled in the art may find that the effective dose for a vaccine administered parenterally may be smaller than a similar vaccine which is administered via drinking water, and the like.

The attenuated strains of the invention and pharmaceutical compositions or vaccines comprising same are highly suitable for immunizing animals such as veterinary species, livestock, and more specifically poultry. For instance, the attenuated Salmonella strains of the invention, and pharmaceutical compositions or vaccines comprising same, are highly suitable for immunizing veterinary species and in particular poultry such as chicken against Salmonellosis and possibly other diseases (e.g. in the case of a multivalent vaccine). The attenuated strains of the invention are particularly suited to protect the animal/veterinary species in question against an attack by the pathogen (the bacterium infecting veterinary species) in question.

A further aspect of the invention therefore concerns a method of immunizing animals, preferably veterinary species, more preferably poultry such as chicken against a disease caused by a bacterium infecting veterinary species, said method comprising the step of: administering to the animal or veterinary species in need thereof an immunizing amount of an attenuated mutant strain of the invention and/or of a vaccine comprising same, whereby a protective immune response is then invoked in the animal or veterinary species. The present invention in particular relates to methods of immunizing veterinary species against Salmonellosis or against an infection by a pathogenic E. coli.

Examples of veterinary species to be immunized against Salmonellosis: poultry, small or heavy livestock such as chicken, turkey, ducks, quails, guinea fowl, pigs, sheep, young calves, cattle etc. An immunizing amount is administered to these animals, preferably via the oral, nasal or parenteral route.

A further aspect of the invention relates to a mutant strain of the invention for use as a medicament (e.g. for use in a vaccine). Yet another aspect of the invention relates to the use of an attenuated mutant strain of the invention for the preparation of a medicament, such as a vaccine, for the prevention (and/or treatment) of a disease caused by a pathogen (the bacterium infecting veterinary species) such as Salmonellosis. Examples of animals or veterinary species to be treated and recommended doses are given above.

Yet another aspect of the invention concerns the use of mutants of the invention, and in particular flagellin mutants, as serological markers to distinguish between vaccinated animals and animals that are naturally infected, id est have been into contact and became infected by a wild-type strain.

The invention for instance relates to a method for a serological distinction between vaccinated animals and animals infected by a wild-type strain, wherein the vaccinated animals have been immunized with a mutant strain wherein a flagellin gene is inactivated, said method comprising the steps of:

Assaying animals for the presence of antibodies raised against flagellin,

Distinguishing infected animals from vaccinated animals based on the presence or absence of said antibodies.

The method of the invention advantageously is an in vitro method. Advantageously animals infected by Salmonellae are as such distinguished from animals that have been immunized with an attenuated live vaccine according to the invention.

Livestock, such as poultry and in particular chicken are known to generate antibodies against flagellin gene products and in particular the FliC gene product. The antibodies in question will thus be detected in an animal infected by a wild-type strain (that generates such antibodies), yet not in an animal that has been vaccinated with a mutant strain wherein a flagellin gene(s) is/are inactivated. The latter do not generate antibodies against e.g. FliC and/or FljB.

The presence of said antibodies is indicative for the presence of wild-type strains and thus infection. The method of the invention thus advantageously allows detection or diagnosis of a Salmonella infection in animals vaccinated by .a mutant strain wherein a flagellin gene(s) is/are inactivated. Such mutant strain may be one of the strains of the invention hereinabove described.

Inactivation of flagellin genes such as fliC, thus allows the use of serological tests, e.g. based on the detection of the FliC protein, for the diagnosis of the presence of wild-type strains, such as wild-type S. enterica, in (vaccinated) animals. In the method of the invention animals are preferably assayed for antibodies against FliC.

The method of the invention is in particular applicable to poultry, more preferably chickens.

### DETAILED DESCRIPTION OF THE INVENTION

It was surprisingly found that the combination of a flagellin mutation (an impairing modification in a gene coding for flagellin; also referred to as a flagellar mutation) and an auxotrophic mutation can lead to highly immunogenic attenuated S. enterica strains. The mutant strains according to the invention carry or comprise a (at least one) modification(s) in a motility gene(s) and a (at least one) modification(s) in a gene(s) involved in the survival or the proliferation of the pathogen in its host.

A gene involved in the survival or proliferation may be a house-keeping gene and/or a virulence gene. Examples of such housekeeping genes and virulence genes can be found in (Mastroeni et al., 2000, The Veterinary Journal 161:132-164, incorporated by reference herein). A preferred example of a house-keeping gene is the guaB gene, yet a modification in an aro, pur, dap, pab, sipC, phoP, phoQ, pagC, cya, and/or crp gene may also be envisaged.

The term “gene” as used herein refers to the coding sequence and its regulatory sequences such as promoter and termination signals.

Such inactivation may be obtained via a deletion by which the gene function is impaired. A person skilled in the art knows how to obtain such mutants and a simple test can tell whether the gene function is impaired. For instance, the mutant strain which fails to express a functional guaB gene product cannot grow on Minimal A medium, unless this medium is supplemented with (e.g. 0.3 mM) guanine, xanthine, guanosine or xanthosine.

Another simple test can tell whether a motility gene function such as a flagellin gene function is impaired. These mutants do not swarm on LB medium containing 0.4% agar.

The modifications in the genes in question should result in attenuation of the mutant strains, preferably at least to a degree that they are suitable for use in a live vaccine.

The Examples below show that additional mutations in a (at least one; one or more) motility gene(s) (fliC, fljB and/or fljBA) advantageously alleviated the residual pathogenicity of a guaB deletion mutant, and improved the protection of the immunized animals against challenge with a lethal dose of wild-type S. enterica.

The flagellar filament of all members of the genus Salmonella is a multimer of a single protein, the flagellin protein (van Asten et al., 1995, Journal of Bacteriology 177:1610-2613).

FliC is the phase 1 filament subunit protein of flagellin (Clacci-Woolwine et al., 1998, Infection; and Immunity 66:1127-1134).

S. Typhimurium has two flagellin genes (fliC and fljB) that are located at different sites of the chromosome and that show phase variation. The promoter of fljB forms part of a chromosomal fragment that can be inverted by site-specific recombination. Depending on the orientation, either fljB is expressed together with fljA, the latter encoding a repressor of the fliC gene; or fliC is brought to expression. E. coli, another member of the Enterobacteriaceae, can also possess two flagellin genes that respectively share homology with the fliC and fljB genes of S. enterica (Tominaga, 2004, Genes Genet. Syst. 79:1-8).

Inactivation of the fliC gene in S. Enteritidis (encoding the major flagellar protein) increased both the safety and effectiveness of a vaccine administered to the inbred mouse line BALB/c. This line is very sensitive to systemic salmonellosis.

This proves amongst others that flagellin is not an essential antigen for the induction of a protective immune response against Salmonella in BALB/c mice despite many indications therefore in literature.

S. Enteritidis flagellin is immunogenic in chickens and carries the H:g,m antigenic determinants (van Asten et al., 1995; Wyant et al., 1999, Infection and Immunity 67:1338-1346; Ogushi et al., 2001, The Journal of Biological Chemistry 276:30521-30526).

There is evidence that flagellae from various species of Gram-negative bacteria (e.g. those of S. Enteritidis and S. typhi) activate monocytes to produce proinflammatory cytokines (e.g. the tumor necrosis factor alpha) and mediate activation of interleukin-1 receptor-associated kinase (IRAK).

It is thus thought that Gram-negative flagellin plays an important and previously unrecognized role in the innate immune response to Gram-negative bacteria. FliC may be of particular importance during the course of infections in the gastrointestinal tract (Clacci-Woolwine et al., 1998; Wyant et al., 1999; Moors et al., 2001, Infection and Immunity 69:4424-4429).

There is a lot of ambiguity in literature as to the degree to which flagella contributes to virulence in poultry and/or humans.

Van Asten et al. (2000, FEMS Microbiol Lett. 185:175-9) have shown that inactivation of the flagellin gene of S. Enteritidis strongly reduces (50-fold) invasion into Caco-2 cells (human colon carcinoma cell line), while the bacterial adherence was not really affected. Said report is limited to in vitro results.

Parker and Guard-Petter (2001, FEMS Microbiology Letters 204:287-291) on the other hand found that, upon oral challenge of chicks, a fliC::Tn10 mutant was equally virulent to the wild-type. This indicates that the presence of flagellin was not necessary to achieve at least a moderate level of invasion after oral challenge. When applied subcutaneously, flagellar mutants were significantly attenuated in comparison to the wild-type strain.

There are thus a lot of indications in the prior art that teach away from constructing e.g. S. enterica double (or triple) mutants according to the invention. Inactivation of one or more motility genes helps reduce the remaining virulence of for instance guaB deletion mutants but there are other advantages as well.

The inactivation of e.g. the fliC gene advantageously allows the use of serological tests, based on the detection of antibodies directed against the FliC protein, for the diagnosis of the presence of wild-type S. enterica, e.g. S. Enteritidis, in the (vaccinated) animals. Immunodetection is possible via ELISA, via RIA techniques and/or any other known immunological test or format.

IDEXX Laboratories has a test on the market (FlockChek® Salmonella Enteritidis Antibody Test Kit) to reliably detect antibodies against H-antigenic determinants of the FliC flagellin of S. Enteritidis (H:g,m flagellar epitopes).

The above demonstrates that double and/or triple S. enterica mutants of the invention, bearing a (impairing) genetic modification in a gene involved in survival or the proliferation of the pathogen in the host and in a gene(s) involved in motility have advantages over attenuated S. enterica strains that are in the art.

The mutant strains of the invention are highly suitable for use in a live attenuated vaccine, as a live vector and/or a DNA-mediated vaccine. The term “vaccine” is meant to include prophylactic as well as therapeutic vaccines. Preferably the vaccine is prophylactic.

“Live vector” vaccines, also called “carrier vaccines” and “live antigen delivery systems”, comprise an exciting and versatile area of vaccinology (Levine et al, 1990, Microecol. Ther. 19:23-32). In this approach, a live viral or bacterial vaccine is modified so that it expresses protective foreign antigens of another microorganism, and delivers those antigens to the immune system, thereby stimulating a protective immune response. Live bacterial vectors that are being promulgated include, among others, attenuated Salmonella.

An object of the invention is to provide attenuated mutant strains for use in a live vaccine, possibly a polyvalent dive vaccine. By a “polyvalent vaccine” or “multivalent vaccine” is meant in particular a vaccine comprising antigenic determinants from a number of different disease-causing organisms.

One of the objects of the invention is therefore to provide a vaccine against e.g. Salmonellosis comprising:

a pharmaceutically effective or an immunizing amount of an attenuated mutant strain of the invention; and

a pharmaceutically acceptable carrier or diluent.

Another object of the invention is to provide a live vector vaccine comprising:

a pharmaceutically effective or an immunizing amount of an attenuated mutant strain of the invention, wherein said mutant encodes and expresses a foreign antigen; and

a pharmaceutically acceptable carrier or diluent.

The particular foreign antigen employed in the live vector is not critical to the present invention.

Still another object of the invention is to provide a DNA-mediated vaccine comprising:

a pharmaceutically effective amount or an immunizing amount of an attenuated mutant strain of the invention; wherein said mutant contains a plasmid which encodes and expresses in a eukaryotic cell, a foreign antigen; and

a pharmaceutically acceptable carrier or diluent.

Details as to the construction and use of DNA-mediated vaccines can be found in U.S. Pat. No. 5,877,159, which is incorporated by reference herein in its entirety. Again, the particular foreign antigen employed in the DNA-mediated vaccine is not critical to the present invention.

The decision whether to express the foreign antigen in the pathogen (using a prokaryotic promoter in a live vector vaccine) or in the cells invaded by the pathogen (using an eukaryotic promoter in a DNA-mediated vaccine) may be based upon which vaccine construction for that particular antigen gives the best immune response in animal studies or in clinical trials, and/or, if. the glycosylation of an antigen is essential for its protective immunogenicity, and/or, if the correct tertiary conformation of an antigen is achieved better with one form of expression than the other (U.S. Pat. No. 5,783,196).

By a “pharmaceutically effective amount” is meant an amount much greater than normal to overcome (prevent and/or treat) the disease in question, e.g. Salmonellosis. By an “immunizing amount” as used herein is in fact meant an amount that is able to induce a (protective) immune response in the animal that receives the pharmaceutical composition/vaccine. The immune response invoked may be a humoral, mucosal, local and/or a cellular immune response. As known in the art the necessary amounts may depend on age, sex, weight and many other factors.

The particular pharmaceutically acceptable carriers or diluents employed. are not critical to the present invention, and are conventional in the art. Examples of diluents include: buffer for buffering against gastric acid in the stomach, such as citrate buffer (pH 7.0) containing sucrose, bicarbonate buffer (pH 7.0) alone, or bicarbonate buffer (pH 7.0) containing ascorbic acid, lactose, and optionally aspartame. Examples of carriers include: proteins, e.g., as found in skimmed milk; sugars; e.g. sucrose; or polyvinylpyrrolidone.

Deletion mutants according to the invention were created via standard homologous recombination techniques, whereby the entire gene(s) or at least part of the genes in question in a first step is replaced by a resistance gene and flanking FRT sites.

Preferably, in a second step, said resistance gene is removed by recombination between the two FRT sites. One FRT site and the priming sites P1 and P2 remain by the molecular mechanism of the recombination* removing the antibiotics resistance gene according to Datsenko and Wanner (2000) (see for instance FIG. 4).

The invention will be described in further details in the following examples and embodiments by reference to the enclosed drawings. Particular embodiments and examples are not in any way intended to limit the scope of the invention as claimed. The rationale of the examples given here for S. enterica are equally well applicable to other (Gram-negative) bacteria infecting veterinary species, more in particular other (Gram-negative) bacteria for poultry such as Pasteurella, (pathogenic) E. coli, etc.

### SHORT DESCRIPTION OF THE DRAWINGS

FIG. 1 gives a schematic overview of the biosynthetic pathway of guanosine monophosphate.

AICAR: 5′-phosphoribosyl-4-carboxamide-5-aminoimidazole; ATP: adenosine triphosphate; G: guanine; GMP: guanosine monophosphate; GR: guanosine; Hx: hypoxanthine; HxR: hypoxanthine riboside (inosine); IMP: Inosine monophosphate; X: Xanthine, XMP: Xanthosine monophosphate; guaA: GMP synthetase, guaB: IMP dehydrogenase; guaC: GMP reductase.

FIG. 2 represents contig 1294 of the S. Enteritidis genome (SEQ ID NO: 19). The ATG initiation codon and TGA termination codon of the guaB gene are in bold. N can be A, C, T or G

FIG. 3 represents the sequence of the ΔguaB fragment of S. Enteritidis cloned in pUC18 (SEQ ID NO: 20). The primers that were used are indicated by horizontal arrows. The fragment generated with primers GuaB6-GuaB7 was cloned in pUC18. The ATG initiation and TGA termination codon of the guaB gene and the CCCGGG SmaI restriction site are indicated in bold.

FIG. 4 represents the nucleotide sequence of the S. Enteritidis PCR fragment, which includes the guaB deletion, obtained using primer GuaB10 (SEQ ID NO: 21). The PCR fragment was amplified with primers GuaB6-GuaB7, using total genomic DNA of the mutant SM20. The remaining FRT site is indicated in bold italic and the P1 and P2 primers by arrows (Datsenko and Wanner, 2000, PNAS 97:6640-6645). The ATG initiation and TGA termination codon of the guaB gene are indicated in bold.

FIG. 5 represents the guaB gene of S. Typhimurium LT2, section 117 of 220 of the complete genome (SEQ ID NO: 22). The ATG initiation codon and TGA termination codon of the guaB gene are in bold.

FIG. 6 shows the nucleotide sequence obtained after sequencing the PCR fragment amplified with primers FliC1-FliC2 on total DNA of the mutants SM73 and SM89, using primer FliC1 and FliC2 (SEQ ID NO: 23). The remaining FRT site is indicated in bold italic, the ATG initiation and TAA stop codons in bold, and P1 and P2 are indicated with arrows.

FIG. 7 shows the nucleotide sequence obtained after sequencing the PCR fragment amplified with primers FljBA6-FljBA5 on total DNA of the S. Typhimurium mutant SM48, using primer FljBA6 (SEQ ID NO: 24). The remaining FRT site is indicated in bold, P1 and P2 are indicated with arrows.

FIGS. 8-11 represent the deposit receipts for SM69, SM73, SM86 and SM89 respectively.

### EXAMPLES

### Example 1

### Auxotrophic Mutation that Affects the guaB Gene

An auxotrophic insertion mutant of a wild type S. Enteritidis was obtained via insertion mutagenesis. Only when supplemented with 0.3 M guanine, xanthine, guanosine or xanthosine could the mutant strain grow on Minimal A medium.

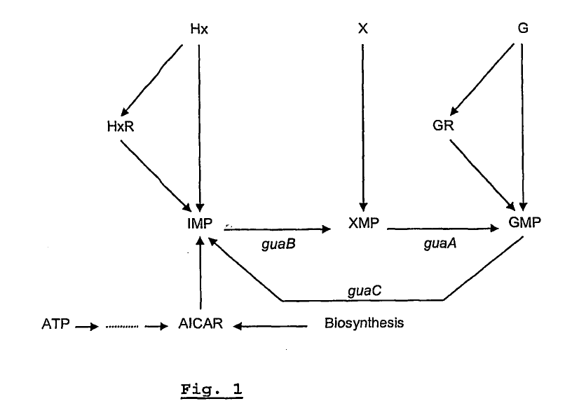

These data strongly suggest that the auxotrophic mutation of the strain affects the guaB gene, encoding the enzyme IMP dehydrogenase (EC 1.1.1.205). This enzyme converts inosine-5′-monophosphate (IMP) into xanthosine monophosphate (XMP) as indicated in FIG. 1.

An insertion mutant can revert, thereby restoring the pathogenicity of the strain. This can limit its applicability in a live attenuated vaccine. In that aspect deletion mutants are preferred. guaB deletion mutants of S. Enteritidis and S. Typhimurium were therefore created and tested. The guaB genes of both serovars are given in FIGS. 2 and 5.

### Example 2

### guaB Deletion Mutants

Construction of guaB Deletion Mutants

A method to generate deletion mutations in the genome of E. coli K12 that was previously published (Datsenko and Wanner, 2000, PNAS 97:6640-6645) was applied for this aim. This method relies on the homologous recombination, mediated by the bacteriophage λ Red recombinase system, of a linear DNA fragment generated by PCR wherein the guaB sequence is substituted by an antibiotic resistance gene. This resistance gene is surrounded by FRT sites and can be excised from the genome by site-specific recombination, mediated by the FLP recombinase.

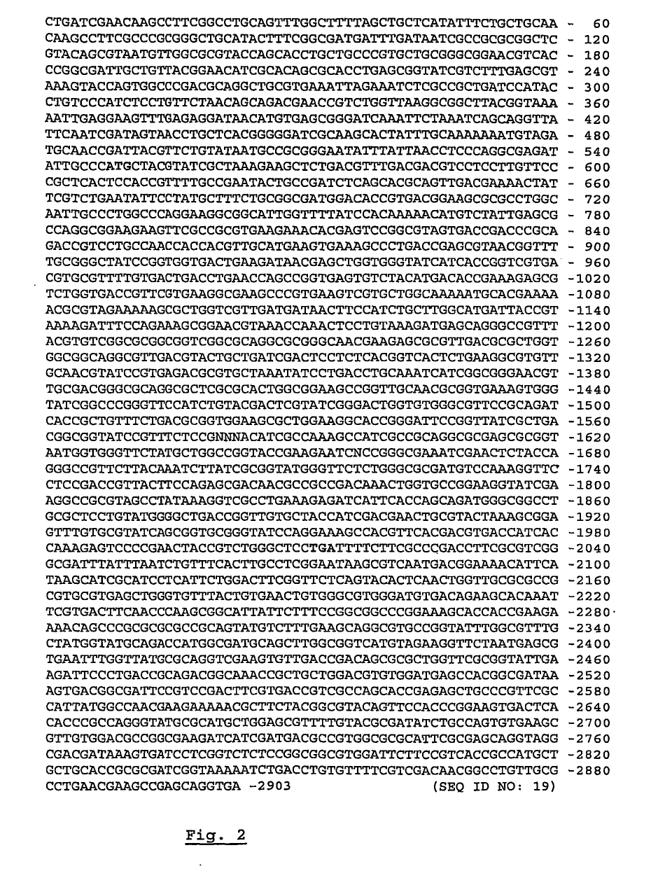

Overlap PCR (Ho et al., 1989, Gene 77:51-59) was applied for the deletion of an internal segment of 861 bp of the guaB coding sequence. The principle relies on the use of two primer sets, GuaB3-GuaB4 (flanking the 5′ end of the guaB gene) and GuaB5-GuaB2 (flanking the 3′ end of the guaB gene). Both sets contain primers (GuaB4 and GuaB5) that are partially complementary and to which a SmaI restriction site was added. After annealing of the resulting complementary sequences and chain elongation, PCR with the outward primers GuaB6 and GuaB7 generated a fragment with a 6 basepair SmaI site replacing an 861 basepair internal segment of the guaB coding sequence. This ΔguaB fragment was cloned in the vector pUC18 (see FIG. 3).

The chloramphenicol resistance gene (cat) with its flanking FRT sequences was amplified using the primers P1 and P2 (Datsenko and Wanner, 2000) and plasmid pKD3 DNA as a template. This PCR fragment was ligated in the SmaI site of the cloned tguaB fragment. The desired fragment was generated using nested primers (GuaB6-GuaB7). The resulting PCR fragment was electroporated into S. Enteritidis 76Sa88 harbouring the temperature sensitive replication plasmid pKD46, encoding the bacteriophage Lambda Red recombinase system. The chloramphenicol resistant transformants were tested on Minimal A medium and on Minimal A medium supplemented with 0.3 mM guanine. The ΔguaB::catFRT mutants were confirmed by PCR using the following primer combinations: GuaB6-GuaB7, GuaB6-P2, GuaB7-P1 and P1-P2.

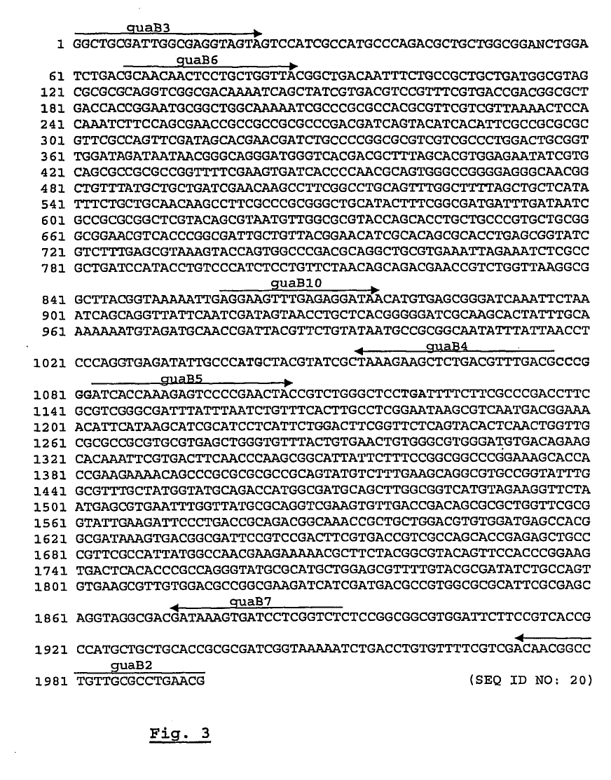

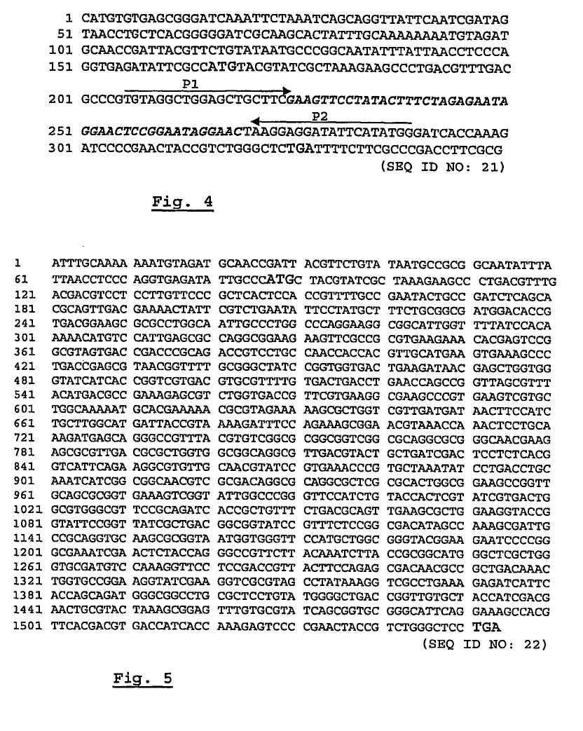

The S. Enteritidis ΔguaB::catFRT mutant (SM12) was electroporated with the temperature sensitive replication plasmid pCP20, encoding the FLP recombinase, to remove the cat gene. The resulting strain S. Enteritidis ΔguaB was named SM20. The PCR fragment in which the deletion is located was obtained using total genomic DNA of the mutant SM20 and the primer combination GuaB6-GuaB7. The ΔguaB mutation was confirmed by sequencing, using the primer GuaB10, of this fragment (see FIG. 4).

The sequences of all above-mentioned primers are given in Table 1.

To avoid the presence of possible additional mutations, caused by the expression of the Red recombinase system, an isogenic strain was constructed.

The ΔguaB::catFRT mutation of the mutant SM12 was transduced with bacteriophage P22 HT int⁻ (Davis, R. W., Botstein D. and Roth, J. R. (1980) In Advanced Bacterial Genetics, A manual for genetic engineering. Cold Spring Harbor Laboratory, Cold Spring Harbor, N.Y.) lysate of SM12 to wild type S. Enteritidis 76Sa88. The cat gene was removed using the plasmid pCP20. The resulting strain S. Enteritidis ΔguaB was called SM69 having deposit number LMG P-21641.

A ΔguaB mutant of S. Typhimurium strain 1491S96 was constructed using the same procedure and the same primers. The resulting strain was named SM19. SM86 (having the deposit number LMG P-21646) is the isogenic strain obtained after transduction of ΔguaB::catFRT to S. Typhimurium strain 1491596 using a bacteriophage P22 HT int⁻ lysate of SM9, and after excision of the cat gene.

The ΔguaB mutants SM19, SM20, SM69 and SM86 are sensitive to bacteriophage P22 HT int⁻. This proves the presence of intact lipopolysaccharides (LPS).

Virulence and Protection Tests with the S. Enteritidis guaB Deletion Mutant SM20 in Mice.

The virulence of the mutant SM20 in mice was tested by oral infection of 6-8 week old female BALE/c mice (Pattery, et al., 1999, Mol. Microbiol. 33(4):791-805) in two independent experiments. These were performed as described above. The wild type strain S. Enteritidis 76Sa88 was tested in parallel as a positive control. The S. Enteritidis 76Sa88 ΔaroA mutant SM50 was included in the experiment as a vaccine control. This mutant carries a precise deletion of the complete aroA coding sequence and was constructed by the method of Datsenko and Wanner (2000).

The complete data are given in Tables 2 and 3. These results demonstrate that the ΔguaB mutant SM20 is strongly attenuated in mice but still shows some residual pathogenicity when administered at this high dose. Oral immunization with this mutant induces protective immunity against infection by a high dose of the corresponding pathogenic wild type S. Enteritidis strain 76Sa88. The protection is at least equal to the protection conferred by the S. Enteritidis ΔaroA mutant SM50.

Virulence and Protection Tests with the Isogenic guaB Deletion Mutants SM69 and SM86 in Mice.

The virulence of the mutants SM69 and SM86 in mice was tested by oral infection of 6-8 week old female BALB/c mice. These were performed as described above. The wild type strains S. Enteritidis 76Sa88 and S. Typhimurium 1491S96 were tested in parallel as positive controls.

The complete data are given in Tables 6, 7, and 10-13. These results demonstrate that the ΔguaB mutants SM69 and SM86 are strongly attenuated in mice, yet still show some residual pathogenicity when administered at this high dose. Oral immunization with the mutants induces protective immunity against infection by a high dose of the corresponding pathogenic wild type strain.

### Example 3

### Flagellin Mutants of S. Enteritidis and S. Typhimurium

It was then tested whether an additional (a further) modification in a motility gene (e.g. a flagellin gene) could further reduce the residual pathogenicity that remained in single mutants like SM20 that carry a deletion mutation in the guaB gene.

S. Enteritidis strains that contain only one gene coding for flagellin, fliC, were used in preliminary experiments. Double mutants were constructed wherein the guaB and fliC genes of S. Enteritidis were inactivated. For S. Typhimurium, double (ΔguaBΔfliC; ΔguaBΔfljBA) and triple (ΔguaBΔfliCΔfljBA) mutants were constructed.

### Construction of Af/iC Mutants (SM24, SM30)

PCR using the FliCP1-FliCP2 primer combination on the template plasmid pKD3 (catFRT) or pKD4 (kanFRT) amplifies the recombinant fragment which contain the antibiotic resistance gene together with the FRT sites and priming sites P1 and P2, and extensions homologous to the initial 50 (1-50) and the terminal (1468-1518) 50 nucleotides of the fliC coding sequence. In this region, S. Typhimurium 1491S96 and S. Enteritidis 76Sa88 show respectively 100% and 98% sequence identity with the primers. The primer FliCP1 contains an additional G at position 37 compared to SEQ ID NO 22. Therefore the ΔfliC mutant allele encodes a 16 amino acid peptide, of which the first 12 amino acids correspond to the amino terminus of FliC. An internal segment of 1416 by (51-1467) of the fliC coding sequence (1-1518) will be substituted.

The resulting PCR product (1 μg) was electroporated to S. Typhimurium 1491S96 (pKD46) and S. Enteritidis 76Sa88 (pKD46), previously induced with 0.2% arabinose, encoding the Lambda recombinase system.

Antibiotic resistant candidate substitution mutants were confirmed by PCR, using primers FliC1 and FliC2 and total DNA of the mutant strains and the wild type strain. Restriction analysis was carried out to distinguish between PCR fragments with approximately the same size. For the restriction of the wild type S. Typhimurium PCR fragment amplified with FliC1-FliC2, the enzyme EcoRV was used. Two fragments (470 bp and 1021 bp) were obtained. The fragment amplified for the fliC substitution mutant doesn't contain an EcoRV restriction site. In case of S. Enteritidis the enzyme ApoI was used. This enzyme cuts the wild type fliC fragment of S. Enteritidis in 2 pieces (345 bp and 1147 bp). The fragment obtained for the fliC substitution mutant doesn't contain an ApoI restriction site.

The motility of the mutants was tested on LB medium (Miller, 1992, A short course in bacterial genetics, a laboratory manual and handbook for Escherichia coli and related bacteria. Cold Spring Harbor Laboratory Press, Cold Spring Harbor, N.Y.) with 0.4% agar. Wild type S. Typhimurium and wild type S. Enteritidis swarm on this medium. The S. Typhimurium fliC substitution mutant swarms out, as the fljB gene is still present in the mutant. The S. Enteritidis fliC substitution mutant doesn't swarm anymore. These results were confirmed by microscopic observation.

Electrocompetent cells of the different mutants were prepared and transformed by electroporation with plasmid pCP20 (extracted from S. Typhimurium χ3730, R. Curtiss, III, S. M. Kelly, P. A. Gulig, C. R. Gentry-Weeks and J. E. Galán. Avirulent Salmonella expressing virulence antigens from other pathogens for use as orally-administered vaccines. In: J. Roth, Editor, Virulence Mechanisms, American Society for Microbiology, Washington D.C. (1988), p. 311) to remove the antibiotic resistance gene. The transformants were incubated at 43° C. This will eliminate the temperature sensitive pCP20 plasmid and should eliminate the antibiotic resistance gene. The loss of the antibiotic resistance gene in S. Typhimurium and S. Enteritidis ΔfliC::catFRT mutants was confirmed.

The deletion mutants, originating from the chloramphenicol resistant substitution mutants, were confirmed by PCR using the primer combination FliC1/FliC2. For both S. Typhimurium ΔfliC and S. Enteritidis ΔfliC a fragment of 185 bp was amplified.

The deletion was confirmed by sequencing the amplified fragments using primer FliC3.

The obtained mutants were tested on LB medium containing 0.4% agar: wild type S. Typhimurium and wild type S. Enteritidis swarms out on this medium, also S. Typhimurium ΔfliC swarms out (fljB flagellar gene is still present). S. Enteritidis ΔfliC as expected doesn't swarm out.

Construction of ΔfljBA Mutants (SM48)

S. Typhimurium contains a second flagellin gene, fljB. This gene is expressed together with fljA, that codes for the repressor of fliC. In the present case, both fljA and fljB were deleted. FljB is 1520 bp long and codes for the protein flagellin. FljA is 539 bp long and codes for the repressor of fliC. The total length of the fragment that was deleted (fljBA): 2127 bp.

Primers were designed which show 51 nucleotides homology with sequences of the fljBA gene and homology with sequences of the template plasmid, which flank the antibiotic resistance gene and FRT sites. Primer FljBAP1 shows homology with the sequence starting from the startcodon of fljB till 51 bp downstream (1-51) and primer FljBAP2 shows homology with the sequence starting from the stop codon of fljA till 51 bp upstream (2076-2127). Primers FljBAP1 and FljBAP2 show homology at their 3′ ends with the priming sites P1 and P2 in the template plasmid flanking the resistance gene with the FRT sites.

PCR using primers FljBAP1 and FljBAP2 (sequences in table 1) and template DNA pKD3 (catFRT) or pKD4 (kanFRT) amplified fragments of the desired length.

1 μg of the PCR product was electroporated to S. Typhimurium, transformed with pKD46 or pKD20. The selected kanamycine and chloramphenicol resistant transformants were confirmed by PCR.

The mutants were tested on LB medium containing 0.4% agar. Wild type S. Typhimurium, S. Typhimurium ΔfljBA::kanFRT and S. Typhimurium ΔfijBA::catFRT swarm out (fliC is still present). Motility of the three strains was confirmed by microscopic observation.

Electrocompetent cells of the different mutants were electroporated with the pCP20 plasmid (originated from S. Typhimurium LT2 restriction mutant χ3730) to remove the antibiotic resistance gene. After 2 hours of incubation at 28° C. the culture was plated on LB medium with carbenicilline. After incubation of the transformants. at 43° C. on LB, they were tested on the loss of the plasmid and the antibiotic resistance gene. The deletion mutations were confirmed by means of PCR and sequencing of the fragment.

PCR using primer combination FljBA6/FljBA5 (sequence in table 1) amplified a fragment of 2112 bp for the wild type S. Typhimurium and a fragment of 185 bp for the S. Typhimurium ΔfljBA mutant SM48.

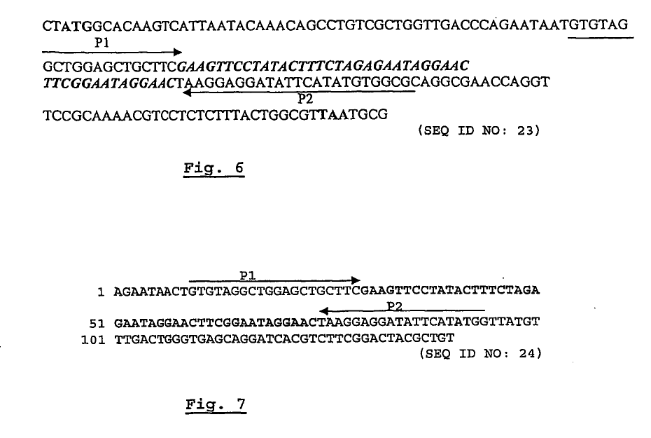

The deletion in mutant SM48 was confirmed by sequencing using primer FljBA6 on the PCR fragment obtained using primers FljBA6-FljBA5 (FIG. 7).

Construction of the S. Typhimurium 1491596 ΔfljBAΔfliC Double Mutant (SM23)

The strain S. Typhimurium ΔfljBA::kanFRT (pKD46) was used to construct the double mutant. Electrocompetent cells were prepared at a temperature of 28° C. (temperature sensitive plasmid pKD46). The electrocompetent cells were electroporated with the recombinant fliC fragment, in which the fliC gene is substituted with the chloramphenicol resistance gene (see earlier examples). To screen and confirm the candidate mutants the procedure used in the construction of the fliC mutant was followed. The desired genotype: S. Typhimurium ΔfljBA::kanFRT ΔfliC::catFRT. To eliminate the antibiotic resistance gene the protocol previously described was followed. The deletions in S. Typhimurium ΔfljBA ΔfliC(SM23) were confirmed by PCR.

The double mutant S. Typhimurium ΔfljBA ΔfliC (SM23) was as expected not motile on LB medium with 0.4% agar. The non-motility of the strain was confirmed by microscopic observation.

### Combination of the Auxotrophic and the Flagellar Mutations

P22-transduction (Davis, R. W., Botstein D. and Roth, J. R. (1980) In Advanced Bacteria/Genetics, A manual for genetic engineering. Cold Spring Harbor Laboratory, Cold Spring Harbor, N.Y.) was used to combine the mutations. P22-lysates of the substitution mutants were used to construct the combined deletion mutants. The transduction was confirmed by PCR (the same protocol and primers were used as in previous confirmations). For the elimination of the antibiotic resistance genes the previously described protocol using the pCP20 helper plasmid was used. The deletions were confirmed by PCR. Mutants constructed this way are: S. Typhimurium ΔfliC AguaB (SM32), S. Typhimurium ΔfljBA ΔguaB (SM35), S. Typhimurium ΔfliC ΔfljBA ΔguaB (SM27) and S. Enteritidis ΔfliC ΔguaB (SM21).

### Construction of Isogenic Deletion Mutants

To exclude the possibility that additional unknown mutations (which can have an effect on the attenuation of the strains) are present in the candidate vaccine strains, dedicated to the use of the method described by Datsenko and Wanner (2000) for the construction of the deletion mutants, isogenic deletion mutants were constructed. The mutations were transduced to a wild type background, by means of P22 phage transduction (Davis, R. W., Botstein D. and Roth, J. R. (1980) In Advanced Bacterial Genetics, A manual for genetic engineering. Cold Spring Harbor Laboratory, Cold Spring Harbor, N.Y.). The antibiotic resistant substitution mutants were used as donor strain. For the elimination of the antibiotic resistance genes and confirmation of the deletions, the same protocols were used as in the previous experiments.

Constructed mutants: S. Enteritidis ΔguaB (SM69, having the deposit number LMG P-21641); S. Enteritidis ΔfliC(SM71); S. Typhimurium ΔguaB (SM86, having the deposit number LMG P-21646); S. Typhimurium ΔfliC (SM91); S. Typhimurium ΔfljBA (SM90), S. Enteritidis ΔguaB ΔfliC(SM73, having the deposit number LMG P-21642); S. Typhimurium ΔguaB ΔfliC(SM104); S. Typhimurium ΔguaB ΔfljBA (SM87); S. Typhimurium ΔfljBA ΔfliC (SM83); S. Typhimurium ΔguaB ΔfljBA ΔfliC (SM89, having the deposit number LMG P-21643).

### Example 4

### Virulence and Protection Experiments with S. Enteritidis Vaccine Strains

Effect of the Inactivation of the fliC Gene on the Virulence of a S. Enteritidis Vaccine Strain.

To study the effect of the inactivation of the fliC gene on the immunogenicity of a S. Enteritidis vaccine strain, two independent virulence and protection tests were carried out in 7 weeks old female BALB/c mice with both mutant SM20 (ΔguaB) and SM 21 (ΔguaB ΔfliC) (Tables 4 and 5).

For the virulence assay, the mice were orally infected with a dose of about 10⁸ CFU, which corresponds to approximately 10⁵ times the LD₅₀ of the wild type strain (Pattery et al., 1999, Molecular Microbiology 33:791-805). The mice were observed during 21 days. All mice inoculated with the wild type S. Enteritidis strain 76Sa88 died within 9 days after infection, while the non-infected control mice remained healthy during the observation period of 21 days. In the first experiment mice infected with the S. Enteritidis ΔguaB mutant SM20 showed typical disease symptoms (reduced activity, untidy coat and curved back) and one out of ten died. In the second experiment no disease symptoms were observed with SM20. The S. Enteritidis l ΔguaB ΔfliC mutant SM21 was asymptomatic in both experiments.

### Efficacy of the Mutants SM20 and SM21 to Confer Protection: Protection Tests

The efficacy of the mutants SM20 and SM21 to confer protection was tested three weeks after the initial immunization by oral challenge with about 10⁵ LD50 of the wild type S. Enteritidis strain 76Sa88 (LD50=10³ CFU). The mice were observed during 21 days. All non-immunized mice died after challenge. In the second experiment, one out of three mice vaccinated with SM20 died. All other vaccinated mice survived the challenge without observable disease symptoms. These data show that both mutants are attenuated and confer protection against challenge with the corresponding wild type strain.

To ascertain that no additional unknown mutations were present, which could contribute to the attenuation of the candidate vaccine strains, the mutations containing the selectable resistance genes were transferred to a wild type background by P22 transduction (Davis, R. W., Botstein D. and Roth, J. R. (1980) In Advanced Bacterial Genetics, A manual for genetic engineering. Cold Spring Harbor Laboratory, Cold Spring Harbor, N.Y.).

Efficacy of the Inactivation of the fliC Gene on the Virulence of S. Enteritidis: Virulence and Protection Tests with Isogenic Strains.

The virulence and protection tests in BALE/c mice were repeated with the isogenic strains SM69 (ΔguaB), SM71 (ΔfliC) and SM73 (ΔguaB ΔfliC) after. confirmation by PCR and phenotypic characterization as earlier described. In this virulence assay, a ΔfliC mutant was included, to study the effect of the inactivation of the fliC gene on the virulence of S. Enteritidis. Similar conditions as described above were used for the virulence and challenge experiments. Data obtained for the transductants SM69 and SM73 (Tables 6 and 7) confirmed the observations made in the earlier experiments.

Comparison between both guaB deletion mutants indicates that the S. Enteritidis ΔguaB ΔfliC double mutant SM73 is more attenuated and confers better protection against challenge with high doses of the wild type strain than the S. Enteritidis ΔguaB single mutant SM69. The virulence assay performed with the S. Enteritidis ΔfliC mutant SM71 showed that this mutant remained as virulent as the wild type strain under the applied conditions.

### Immunological Responses and Antibody Production

Fifty-four days following the initial immunization in the first experiment, blood samples were collected from the tail artery of the mice. Anti-lipopolysaccharide (LPS) IgG titers were determined by means of enzyme-linked immunosorbent assay (ELISA) using S. Enteritidis LPS (Sigma) for coating. Comparison between sera of mice immunized with SM20 and SM21 showed that in both cases anti-LPS serum IgG responses were elicited and that no significant differences in titer were measured. Oral immunization with a second and third dose, 66 and 95 days after the initial immunization did not enhance the anti-LPS IgG levels in serum (data not shown).

### Example 5

### Virulence and Protection Experiments with S. Typhimurium Transductants

Virulence and protection experiments were carried out with the transductants in 7 weeks old female BALB/c mice. The mice were orally inoculated with approximately 10⁸ cells. The mice were observed daily for a period of 21 days. After this period, the mice were challenged with 10⁸ cells of the wild type pathogenic strain, and the mice were observed over a period of 21 days.

Symptoms of disease and the survival rate are noted in Tables 10-13. The single and double flagellar mutants remained highly virulent when orally administered, all mice died. The mice inoculated with the guaB mutant showed mild symptoms of diseases (mice had been fighting, only two remained alive). The combined ΔguaB ΔfljBA, ΔguaB ΔfliC and ΔguaB ΔfliC ΔfljBA mutants were highly attenuated. No symptoms of disease were observed during the 21 days observation period after vaccination. These mutants conferred good protection after challenge with a high dose of the wild type strain. Only a reduced activity of the mice after challenge can be observed.

These results also show that the flagellar mutations do not affect the immunogenic capacities of the strains when administered to BALB/c mice. The flagellar mutations can be useful as a serological marker to distinguish between the vaccine strain and the wild type strain. Combination of the auxotrophic mutation with the flagellar mutation(s) gives the best results concerning the reduced virulence of the mutants in mice and the protection against the corresponding wild type strain.

The attenuation of the S. Typhimurium ΔguaB ΔfliC ΔfljBA triple deletion mutant and the S. Typhimurium double deletion mutants, ΔguaB ΔfljBA and ΔguaB ΔfliC, were comparable.

### Example 6

### Safety Evaluation of S. enteritidis Vaccine Strains

### Safety Evaluation SM69 in Chicks Inoculated at the Age of One Day by the Intratracheal or Oral Gavage Route

The objective of this study was to evaluate the safety of S. Enteritidis ΔguaB mutant strain SM69 master seed in one-day-old chickens. Mortality was used as a primary parameter for the determination of safety.

Chicks at one day of age were leg-banded and randomly placed in each of the four treatment groups (Group 1: SM69-IT, group 2: SM69-OG, group 3: PBS-IT and Group 4: PBS-OG). After the master seed inoculation, the birds from groups 1 and 2 were placed in one isolator and those of groups 3 and 4 in another isolator.

Chickens in groups 1 and 2 were inoculated with the SM69 master seed by the intratracheal (IT) route or oral gavage (OG) route, respectively, with an actual titer of 1.3×10⁸ CFU/0.2 ml per bird. Chickens in groups: 3 and 4 were administered with 0.2 ml PBS (phosphate buffered saline) per bird by the intratracheal or oral gavage, respectively.

Following the inoculation of SM69 or PBS, chick mortality was observed daily until 38 days post inoculation. Table 8 summarizes the results of mortality for all 4 groups. In group 1, one bird died during the inoculation due to inoculation trauma. Two birds died at 2 days post inoculation (DPI). Three birds died from day 3 to day 13 (at 3, 5 and 13 DPI respectively). A total of 6 birds thus died in group 1. In group 2, two birds died in total. One died due to inoculation trauma and one died at day 5 post inoculation. No birds died in the PBS treated groups either by the intratracheal or oral gavage route.

This study indicates that the S. Enteritidis ΔguaB mutant strain SM69 is not safe when administered at 1.3×10⁸ CFU per bird at one day of age by the intratracheal or oral gavage route.

### Safety Evaluation of SM69 in Chicks Inoculated at the Age of 2 Weeks by the Intratracheal or Oral Gavage Route

Safety of the S. Enteritidis ΔguaB mutant strain SM69 was then evaluated in 2 week-old SPF chickens by the intratracheal and oral gavage routes. Mortality was used as a primary criterion and body weight as a secondary criterion for the determination of safety.

Birds at 2 weeks of age were leg-banded and randomly placed in each of the four treatment groups: SM69-IT, SM69-OG, Poulvac ST-IT and PBS-IT. Ten birds in group 1 were inoculated with SM69 by the intratracheal route; ten birds in group 2 were inoculated with SM69 by oral gavage; ten birds in group 3 were inoculated with a Salmonella Typhimurium AroA⁻ vaccine (Poulvac® ST) by the intratracheal route; and five birds in group 4 were inoculated with PBS by the intratracheal route.

Chickens in groups 1 and 2 were inoculated with SM69 master seed by the intratracheal or oral gavage route, respectively, with the actual titer of 2.3×10⁸ CFU/0.2 ml per bird. Chickens in group 3 were administered with Poulvac® ST by the intratracheal route with 2.2×10⁸ CFU/0.2 ml per bird. Chickens in group 4 were administered by the intratracheal route with 0.2 ml PBS per bird.

After inoculation, the birds from treatment groups 1 and 2 were placed in one isolator and those from groups 3 and 4 in another isolator.

Following inoculations, mortality was observed daily until 21 days post-inoculation. Body weight of all birds was also recorded at the end of the study period (21 days). Poulvac® ST and PBS were used as intratracheal procedure controls.

During the 21-day observation period, one bird in the SM69 intratracheal treatment group (group 1) died from an infected yolk sac. No mortality was associated with SM69 inoculation, indicating that the SM69 strain is safe at the titer tested, 2.3×10⁸ CFU per bird by the intratracheal and oral gavage routes. As expected, no death was observed either in the Poulvac® ST treated birds at the titer of 2.2×10⁸ CFU per bird or in the PBS treated birds, indicating that the study was valid (Table 9).

Body weight was compared amongst groups in an analysis of variance (ANOVA) model with body weight as the dependent variable and treatment included as an independent variable. Group comparisons were made using Tukey's test for multiple comparisons. The level of significance was set at p<0.05. The study was considered valid because the control chickens (PBS control group) remained healthy and free of clinical signs of diseases or mortality throughout the study.

There were no significant differences in the final body weight in chickens administered with SM69 by the intratracheal or oral gavage inoculation, Poulvac® ST, or PBS (Table 9). Even though no baseline was established of the birds in each group at one day of age, it was unlikely that there was a significant difference in the initial body weight amongst the four groups since the birds were randomly placed into each of the 4 treatment groups.

It can be concluded from the present experiment that SM69 is safe when administered at the tested titer of 2.3×10⁸ CFU per bird at 2 weeks of age by either the intratracheal or oral gavage route.

### Safety Evaluation of SM73 in Chicks Inoculated at the Age of One Day by the Intratracheal or Oral Gavage Route

Safety of the S. Enteritidis deletion mutant strain SM73 (ΔguaB ΔfliC) was evaluated in chickens by the intratracheal and oral gavage routes. Mortality was used as a primary criterion and body weight as a secondary criterion for the determination of safety.

All birds were leg-banded and randomly assigned to one of the four groups of birds included in this study (Group 1: SM73-IT, group 2: SM73-OG, group 3: Poulvac ST-IT and Group 4: PBS-IT). Ten birds in group 1 were inoculated with SM73 by the intratracheal route; ten birds in group 2 were inoculated with SM73 by oral gavage; ten birds in group 3 were inoculated with a S. Typhimurium AroA⁻ vaccine (Poulvac® ST) by the intratracheal route; and five birds in group 4 were inoculated with PBS by the intratracheal route. A total of 4 isolators was used (one for each group) in which the chickens were housed for the duration of the study.

Chickens were inoculated at one day of age. Chickens in groups 1 and 2 were inoculated with SM73 master seed by the intratracheal or oral gavage route, respectively, with an actual titer of 2.5×10⁷ CFU/0.2 ml per bird. Chickens in group 3 were administered with Poulvac® ST by the intratracheal route with 2.1×10⁷ CFU/0.2 ml per bird. Chickens in group 4 were administered by the intratracheal route with 0.2 ml PBS per bird.

Mortality was observed and body weight recorded as described above for SM69. Poulvac® ST and PBS were once again used as intratracheal procedure controls.

During the 21-day observation period, no mortality was recorded for any bird at all. There was further no difference in the final body weight of PBS-IT, Poulvac ST-IT and SM73-OG inoculated birds. The average body weight of the SM73-IT groups was significantly lower in comparison to the SM73-OG group (p=0.0009) but this is most probably due to an experimental error.

It can be concluded from the present experiment that SM73 is safe when administered at the tested titer of a 2.5×10⁷ CFU per bird at one day of age by the intratracheal or oral gavage route.

A deposit has been made according to the Budapest Treaty at the BCCM/LMG Culture Collection, Laboratorium voor Microbiologie, K. L. Ledeganckstraat 35, B-9000 Gent (Belgium) for the following micro-organisms: Salmonella Enteritidis SM69 under deposit number LMG P-21641 (deposit date: 9 Aug., 2002); S. Enteritidis SM73 under deposit number LMG P-21642 (deposit date: 9 Aug., 2002), S. Typhimurium SM86 under deposit number LMG P-21646 (deposit date: 28 Aug., 2002) and S. Typhimurium SM89 under deposit number LMG P-21643 (deposit date: 9 Aug., 2002). The deposits have been made in the name of Prof. J.-P. Hernalsteens, previous address: Vrije Universiteit Brussel, Laboratorium Genetische Virologie, Paardenstraat 65, B-1640 Sint-Genesius-Rhode, current address: Vrije Universiteit Brussel, Onderzoeksgroep Genetische Virologie, Pleinlaan 2, B-1050 Brussels, Belgium.

TABLE 1
Primer sequences

| SEQ | | |
|---|---|---|
| ID | | |
| NO | Primer | Sequence |
| 1 | GuaB2 | 5′ CGTTCAGGCG CAACAGGCCG TTGT 3′ |
| 2 | GuaB3 | 5′ GGCTGCGATT GGCGAGGTAG TA 3′ |
| 3 | GuaB4 | 5′ GGTGATCCCG GGCGTCAAAC GTCAGGGCTT |
| | | CTTTA 3′ |
| 4 | GuaB5 | 5′ TTGACGCCCG GGATCACCAA AGAGTCCCCG |
| | | AACTA 3′ |
| 5 | GuaB6 | 5′ GCAACAACTC CTGCTGGTTA 3′ |
| 6 | GuaB7 | 5′ AGACCGAGGA TCACTTTATC 3′ |
| 7 | GuaB10 | 5′ AGGAAGTTTG AGAGGATAA 3′ |
| 8 | P1 | 5′ GTGTAGGCTG GAGCTGCTTC 3′ |
| 9 | P2 | 5′ CATATGAATA TCCTCCTTAG 3′ |
| 10 | F1iCP1 | 5′ ATGGCACAAG TCATTAATAC AAACAGCCTG |
| | | TCGCTGGTTG ACCCAGAATA ATGTGTAGGC |
| | | TGGAGCTGCT TC 3′ |
| 11 | FliCP2 | 5′ CGCATTAACG CAGTAAAGAG AGGACGTTTT |
| | | GCGGAACCTG GTTMGCCTGC GCCACATATG |
| | | AATATCCTCC TTAG 3′ |
| 12 | F1iC1 | 5′ ATGGCACAAG TCATTAATAC AAACAG 3′ |
| 13 | F1iC2 | 5′ CGCATTAACG CAGTAAAGAG AGGAC 3′ |
| 14 | F1iC3 | 5′ TATCGGCAAT CTGGAGGCAA 3′ |
| 15 | F1jBAP1 | 5′ ATGGCACAAG TAATCAACAC TAACAGTCTG |
| | | TCGCTGCTGA CCCAGAATAA CTGTGTAGGC |
| | | TGGAGCTGCT TC 3′ |
| 16 | FljBAP2 | 5′ TTATTCAGCG TAGTCCGAAG ACGTGATCCT |
| | | GCTCACCCAG TCAAACATAA CCATATGAAT |
| | | ATCCTCCTTA G 3′ |
| 17 | FljBA5 | 5′ CAGCGTAGTC CGAAGACGTG ATC 3′ |
| 18 | FLjBA6 | 5′ ACACTAACAG TCTGTCGCTG CT 3′ |

TABLE 2
Virulence test in BALB/c mice of the S. Enteritidis ΔguaB mutant SM20

| Infection | | | Day of | |
|---|---|---|---|---|
| Strain | Dose | Survival | death | State of the mice |

First Experiment

| | | | | |
|---|---|---|---|---|
| Negative control: milk | / | 11/11 | / | No disease symptoms |
| Positive control: S. Enteritidis 76Sa88 | 2.1 × 10⁸ | 0/5 | 7, 7, 8, | |
| | | | 8, 9 | |
| Vaccine control: S. Enteritidis ΔaroA SM50 | 2.5 × 10⁸ | 10/10 | / | No disease symptoms |
| S. Enteritidis ΔguaB SM20 | 5.1 × 10⁸ | 9/10 | 13 | Disease symptoms between the |
| | | | | 7^{th} and the 14^{th} day after |
| | | | | infection |

Second Experiment

| | | | | |
|---|---|---|---|---|
| Negative control: milk | / | 4/4 | / | No disease symptoms |
| Positive control: S. Enteritidis 76Sa88 | 1.4 × 10⁸ | 0/3 | 8, 9, 9 | |
| Vaccine control: S. Enteritidis ΔaroA SM50 | 2.1 × 10⁸ | 3/3 | / | No disease symptoms |
| S. Enteritidis ΔguaB SM20 | 1.9 × 10⁸ | 3/3 | / | No disease symptoms |

TABLE 3
Challenge of mice vaccinated with the S. Enteritidis guaB mutant SM20

| Vaccination | | Challenge | | | Day of | |
|---|---|---|---|---|---|---|
| Strain | Dose | Strain | Dose | Survival | death | State of the mice |

First Experiment

| | | | | | | |
|---|---|---|---|---|---|---|
| Negative | / | negative | / | 6/6 | / | No disease symptoms |
| control: milk | | control: milk | | | | |
| Negative | / | S. Enteritidis | 1.5 × 10⁸ | 0/5 | 7, 8, 8, | Disease symptoms starting on |
| control: milk | | 76Sa88 | | | 8, 9 | the 5^{th} day after challenge |
| Vaccine control: | 2.5 × 10⁸ | S. Enteritidis | 1.5 × 10⁸ | 3/5 | 9, 13 | Disease symptoms between the |
| S. Enteritidis | | 76Sa88 | | | | 7^{th} and the 14^{th} day after |
| ΔaroA SM50 | | | | | | challenge |
| S. Enteritidis | 5.1 × 10⁸ | S. Enteritidis | 1.5 × 10⁸ | 5/5 | / | Mice are less active between |
| ΔgucB SM20 | | 76Sa88 | | | | the 11^{th} and the 14^{th} day |
| | | | | | | after challenge. |

Second Experiment

| | | | | | | |
|---|---|---|---|---|---|---|
| Negative | / | negative | / | 2/2 | / | No disease symptoms |
| control: milk | | control: milk | | | | |
| Negative | / | S. Enteritidis | 1.5 × 10⁸ | 0/2 | 9, 18 | |
| control: milk | | 76Sa88 | | | | |
| Vaccine control: | 2.1 × 10⁸ | S. Enteritidis | 1.5 × 10⁸ | 1/3 | 9, 21 | Disease symptoms between the |
| S. Enteritidis | | 76Sa88 | | | | 7^{th} and the 21^{st} day after |
| ΔaroA SM50 | | | | | | challenge. |
| S. Enteritidis | 1.9 × 10⁸ | S. Enteritidis | 1.5 × 10⁸ | 2/3 | 10 | Disease symptoms starting on |
| ΔguaB SM20 | | 76Sa88 | | | | the 9^{th} day after infection. |

TABLE 4
Virulence test in BALB/c mice of the S. Enteritidis mutants SM20 and SM21

| Infection | | | Day of | |
|---|---|---|---|---|
| Strain | Dose | Survival | death | State of the mice |

First Experiment

| | | | | |
|---|---|---|---|---|
| Negative control: not infected | / | 11/11 | / | Asymptomatic |
| Positive control: S. Enteritidis 76Sa88 | 2.1 × 10⁸ | 0/5 | 7, 7, 8, | Severe symptoms from day 5 |
| | | | 8, 9 | onwards |
| S. Enteritidis ΔguaB SM20 | 5.1 × 10⁸ | 9/10 | 13 | Mild to severe symptoms from |
| | | | | day 7 till day 17 |
| S. Enteritidis ΔguaB: ΔfliC SM21 | 4.3 × 10⁸ | 10/10 | / | Asymptomatic |

Second Experiment

| | | | | |
|---|---|---|---|---|
| Negative control: not infected | / | 4/4 | / | Asymptomatic |
| Positive control: S. Enteritidis 76Sa88 | 1.4 × 10⁸ | 0/3 | 8, 9, 9 | Severe symptoms from day 6 |
| | | | | onwards |
| S. Enteritidis ΔguaB SM20 | 1.9 × 10⁸ | 3/3 | / | Asymptomatic |
| S. Enteritidis ΔguaB ΔfliC SM21 | 3.2 × 10⁸ | 3/3 | / | Asymptomatic |

TABLE 5
Challenge of mice vaccinated with the S. Enteritidis mutants SM20 and SM21

| Vaccination | | Challenge | | | Day of | |
|---|---|---|---|---|---|---|
| Strain | Dose | Strain | Dose | Survival | death | State of the mice |

First Experiment

| | | | | | | |
|---|---|---|---|---|---|---|
| Negative | / | S. Enteritidis | 1.6 × 10⁸ | 0/5 | 7, 8, 8, | Severe symptoms onwards from |
| control: milk | | wild type | | | 8, 9 | day 6 |
| | | strain 76Sa88 | | | | |
| S. Enteritidis | 2.1 × 10⁸ | S. Enteritidis | 1.6 × 10⁸ | / | / | / |
| wild type strain | | wild type | | | | |
| 76Sa88 | | strain 76Sa88 | | | | |
| S. Enteritidis | 5.1 × 10⁸ | S. Enteritidis | 1.6 × 10⁸ | 4/4 | / | Asymptomatic |
| ΔguaB SM20 | | wild type | | | | |
| | | strain 76Sa88 | | | | |
| S. Enteritidis | 4.3 × 10⁸ | S. Enteritidis | 1.6 × 10⁸ | 5/5 | / | Asymptomatic |
| ΔguaB ΔfliC SM21 | | wild type | | | | |
| | | strain 76Sa88 | | | | |

Second Experiment

| | | | | | | |
|---|---|---|---|---|---|---|
| Negative | / | S. Enteritidis | 1.5 × 10⁸ | 0/2 | 9, 18 | Severe symptoms onwards from |
| control: milk | | wild type | | | | day 6 |
| | | strain 76Sa88 | | | | |
| S. Enteritidis | 1.4 × 10⁸ | S. Enteritidis | 1.5 × 10⁸ | / | / | / |
| wild type strain | | wild type | | | | |
| 76Sa88 | | strain 76Sa88 | | | | |
| S. Enteritidis | 1.9 × 10⁸ | S. Enteritidis | 1.5 × 10⁸ | 2/3 | 10 | No symptoms, except one with |
| ΔguaB SM20 | | wild type | | | | severe symptoms |
| | | strain 76Sa88 | | | | |
| S. Enteritidis | 3.2 × 10⁸ | S. Enteritidis | 1.5 × 10⁸ | 3/3 | / | Asymptomatic |
| ΔguaB ΔfliC SM21 | | wild type | | | | |
| | | strain 76Sa88 | | | | |

TABLE 6
Virulence test in BALB/c mice of the S. Enteritidis mutants
SM71, SM73 and SM69

| Infection | | | Day of | |
|---|---|---|---|---|
| Strain | Dose | Survival | death | State of the mice |
| Negative control: | / | 4/4 | / | Asymptomatic |
| not infected | | | | |
| Positive control: | 3.7 × 10⁸ | 0/3 | 7, 8, 9 | Severe symptoms |
| S. Enteritidis 76Sa88 | | | | onwards from |
| | | | | day 5 |
| S. Enteritidis | 1.4 × 10⁸ | 0/3 | 6, 8, 8 | Severe symptoms |
| ΔfliC SM71 | | | | onwards from |
| | | | | day 4 |
| S. Enteritidis | 7.6 × 10⁸ | 5/5 | / | Mild symptoms, |
| ΔguaB SM69 | | | | from day 11 |
| | | | | till day 18 |
| S. Enteritidis | 1.2 × 10⁸ | 5/5 | / | Reduced activity, |
| ΔguaB ΔfliC SM73 | | | | from day |
| | | | | 11 till day 13 |

TABLE 7
Challenge of mice vaccinated with the S. Enteritidis mutants SM71, SM73 and SM69

| Vaccination | | Challenge | | | Day of | |
|---|---|---|---|---|---|---|
| Strain | Dose | Strain | Dose | Survival | death | State of the mice |
| Negative | / | S. Enteritidis | 3.1 × 10⁸ | 0/4 | 8, 8, 8, | Severe symptoms onwards from |
| control: milk | | wild type | | | 9 | day 5 |
| | | strain 76Sa88 | | | | |
| S. Enteritidis | 3.7 × 10⁸ | S. Enteritidis | 3.1 × 10⁸ | / | / | / |
| wild type strain | | wild type | | | | |
| 76Sa88 | | strain 76Sa88 | | | | |
| S. Enteritidis | 1.4 × 10⁸ | S. Enteritidis | 3.1 × 10⁸ | / | / | / |
| ΔfliC SM71 | | wild type | | | | |
| | | strain 76Sa88 | | | | |
| S. Enteritidis | 7.6 × 10⁸ | S. Enteritidis | 3.1 × 10⁸ | 2/5 | 8, 8, 19 | Severe symptoms onwards from |
| ΔguaB SM69 | | wild type | | | | day 5 |
| | | strain 76Sa88 | | | | |
| S. Enteritidis | 1.2 × 10⁸ | | | 5/5 | / | Asymptomatic |
| ΔguaB ΔfliC SM73 | | | | | | |

TABLE 8
Safety evaluation of the Salmonella mutant SM69 in one-day-old chickens

| Infection | | | | | | |
|---|---|---|---|---|---|---|
| Strain | Group | N | Titer | Route | Survival | Day of death (DPI) |
| S. Enteritidis SM69 | 1: SM69-IT | 10 | 1.3 × 10⁸ | intratracheal | 4/10* | 0, 2, 3, 5, 13 |
| | | | CFU/0.2 ml | | | |
| S. Enteritidis SM69 | 2: SM69-OG | 10 | 1.3 × 10⁸ | oral gavage | 8/10** | 0, 5 |
| | | | CFU/0.2 ml | | | |
| Negative control: PBS | 3: PBS-IT | 10 | PBS - 0.2 ml | intratracheal | 10/10 | — |
| Negative control: PBS | 4: PBS-OG | 10 | PBS - 0.2 ml | oral gavage | 10/10 | — |

IT Intratracheal
OG Oral gavage
DPI Days post inoculation
*In group 1, 1 bird died during inoculation; 1 bird died at 3, 5 and 13 DPI; and 2 birds at 2 DPI respectively
**In group 2, 1 bird died during inoculation; and 1 bird died at 5 days DPI

TABLE 9
Safety evaluation of the Salmonella mutant SM69 in 2-week-old chickens

| | | | | | | Day of | Mean | |
|---|---|---|---|---|---|---|---|---|
| Infection | | | | | | death | weight | Std |
| Strain | Group | N | Titer | Route | Survival | (DPI) | (kg) | weight |
| S. Enteritidis SM69 | 1: SM69-IT | 10 | 2.3 × 10⁸ | IT | 9/10* | 13 | 0.429 | 0.064 |
| | | | CFU/0.2 ml | | | | | |
| S. Enteritidis SM69 | 2: SM69-OG | 10 | 2.3 × 10⁸ | OG | 10/10 | — | 0.420 | 0.044 |
| | | | CFU/0.2 ml | | | | | |
| Vaccine control: Poulvac ® | 3: Poulvac-IT | 10 | 2.2 × 10⁸ | IT | 10/10 | — | 0.423 | 0.046 |
| ST* | | | CFU/0.2 ml | | | | | |
| Negative control: PBS | 4: PBS-IT | 5 | PBS - 0.2 | OG | 5/5 | — | 0.388 | 0.019 |
| | | | mL | | | | | |

IT Intratracheal
OG Oral gavage
DPI Days post inoculation
*Death due to yolk sac infection
**A live S. Typhimurium AroA⁻ vaccine against S. Typhimurium

TABLE 10
Virulence experiments with S. Typhimurium mutant strains in
BALB/c mice. Oral inoculation with approximately 10⁸ cells
of the S. Typhimurium strain 1491S96

| Infection | | |
|---|---|---|
| Strain | Survival | |
| (S. Typhimurium 1491S96) | (day of death) | State of the mice |
| Wild type SM2 | 0/3 | Disease symptoms |
| | (9, 9, 10) | from day 4 onwards |
| ΔfliC SM91 | 0/5 | Disease symptoms |
| | (9, 10, 11, 15, 15) | from day 5 onwards |
| ΔfljBA SM90 | 0/5 | Severe disease symptoms |
| | (8, 10, 10, 11, 34) | from day 7 onwards |
| ΔfljBA ΔfliC SM83 | 0/5 | Severe disease symptoms |
| | (8, 9, 12, 14, 15) | from day 7 onwards |
| ΔguaB SM86 | 2/5* | Mild disease symptoms |
| | (2, 2, 2) | from day 9 |
| | | until challenge |
| ΔguaB ΔfljBA SM87 | 5/5 | No symptoms |
| ΔguaB ΔfliC ΔfljBA SM89 | 5/5 | No symptoms |
| Control (not infected) | 4/4 | No symptoms |

*died after a fight

TABLE 11
Challenge experiments with S. Typhimurium mutant strains in BALB/c mice.
Oral vaccination with approximately 10⁸ cells of the S. Typhimurium
strain 1491S96

| | Challenge | | | |
|---|---|---|---|---|
| Vaccination | Strain | | | |
| Strain | (S. Typhimurium | Dose | Survival | |
| (S. Typhimurium 1491S96) | 1491S96) | (CFU) | (day of death) | State of the mice |
| ΔguaB SM86 | S. Typhimurium 1491S96 | 1.3 × 10⁷ | 2/2 | Mild symptoms until days |
| | | | | 14, afterwards one mouse |
| | | | | showed clear symptoms, the |
| | | | | other was healthy again |
| ΔguaB ΔfljBA SM87 | S. Typhimurium 1491S96 | 1.3 × 10⁷ | 5/5 | Reduced activity between |
| | | | | day 8 and day 10 |
| ΔguaB ΔfliC ΔfljBA SM89 | S. Typhimurium 1491S96 | 1.3 × 10⁷ | 5/5 | Reduced activity between |
| | | | | day 8 and day 10 |
| Control (not infected) | S. Typhimurium 1491S96 | 1.3 × 10⁷ | 0/4 | Severe symptoms from day 6 |
| | | | | onwards |

TABLE 12
Virulence experiments with S. Typhimurium 1491S96
mutant strains in BALB/c mice.

| Infection | | | |
|---|---|---|---|
| Strain | | | |
| (S. Typhimurium | | Survival | |
| 1491S96) | Dose | (day of death) | State of the mice |
| Wild type SM2 | 0.8 × 10⁸ | 1/4 | Disease symptoms |
| | | (11, 13, 14) | from day 6 onwards |
| ΔguaB SM86 | 0.8 × 10⁸ | 5/5 | Weak symptoms |
| | | | on day 13 and 14 |
| ΔguaB ΔfliC SM91 | 2.5 × 10⁸ | 5/5 | No symptoms |
| ΔguaB ΔfljBA SM87 | 1.5 × 10⁸ | 5/5 | No symptoms |
| ΔguaB ΔfliC | 1.7 × 10⁸ | 5/5 | No symptoms |
| ΔfljBA SM89 | | | |
| Control (not infected) | — | 5/5 | No symptoms |

TABLE 13
Protection experiments with S. Typhimurium 1491S96 mutant strains in BALB/c mice.

| Vaccination | | Challenge | | Survival | |
|---|---|---|---|---|---|
| Strain | | Strain | | (day of | |
| (S. Typhimurium 1431S96) | Dose | (S. Typhimurium 1491S96) | Dose | death) | State of the mice |
| ΔguaB SM86 | 0.8 × 10⁸ | S. Typhimurium 1491S96 | 2.7 × 10⁸ | 5/5 | Reduced activity |
| | | | | | from day 6 till day |
| | | | | | 16 |
| ΔguaB ΔfliC SM91 | 2.5 × 10⁸ | S. Typhimurium 1491S96 | 2.7 × 10⁸ | 5/5 | Reduced activity |
| | | | | | onwards from day 7; |
| | | | | | weak symptoms from |
| | | | | | day 8 untill day 14 |
| ΔguaB ΔfljBA SM87 | 1.5 × 10⁸ | S. Typhimurium 1491S96 | 2.7 × 10⁸ | 3/5 | Weak symptoms |
| | | | | (10, 28) | between day 6 and |
| | | | | | day 16 |
| ΔguaB ΔfliC•ΔfljBA SM89 | 1.7 × 10⁸ | S. Typhimurium 1491S96 | 2.7 × 10⁸ | 4/5 | Reduced activity |
| | | | | (19) | between day 8 and |
| | | | | | day 16 |
| Control (not infected) | — | S. Typhimurium 1491S96 | 2.7 × 10⁸ | 0/5 | Severe symptoms from |
| | | | | (8, 9, 10, 11, 16) | day 6 onwards |

1. An attenuated mutant strain of a bacterium infecting veterinary species, wherein said mutant contains at least one first genetic modification and at least one second genetic modification, wherein said first modification is in one or more motility genes, and wherein said second modification is in one or more genes involved in the survival or proliferation of the bacterium in the host.

2. The mutant strain of claim 1, wherein the veterinary species is poultry.

3. The mutant strain of claim 1, which is a Salmonella enterica or Escherichia coli strain.

4. The mutant strain of claim 1, wherein the motility gene encodes flagellin.

5. The mutant strain of claim 4, wherein said mutation is in the flic and/or fljB or fljBA genes.

6. The mutant strain of claim 4, wherein said strain is incapable of swarming out on LB medium containing 0.4% agar.

7. The mutant strain of claim 1, wherein the gene involved in the survival of the bacterium is a house-keeping gene or a virulence gene.

8. The mutant strain of claim 7 , wherein the housekeeping gene that is inactivated is the guaB gene.

9. The mutant strain of claim 8 , wherein the mutant strain contains a deletion mutation that impairs the guaB gene function.

10. The mutant strain of claim 8, wherein said strain is incapable of forming de novo guanine nucleotides.

11. The mutant strain of claim 1, wherein said mutant strain encodes and expresses a foreign antigen.

12. The mutant strain of claim 1, which is an attenuated S. Enteritidis or a S. Typhimurium strain.

13. The mutant strain of claim 1, wherein the genetic modifications are introduced into parent strain S. Enteritidis phage type 4 strain 76Sa88.

14. The mutant strain of claim 13, which is the attenuated S. Enteritidis strain SM73 having the deposit number LMG P-21642.

15. The mutant strain of claim 1, wherein the genetic modifications are introduced into parent strain S. Typhimurium 1491 S96.

16. The mutant strain of claim 15, which is the attenuated S. Typhimurium strain SM89 having the deposit number LMG P-21643.

17. The mutant of claim 1, containing a genetic modification in the guaB gene and a genetic modification in the flue gene.

18. The mutant of claim 1, containing a genetic modification in a guaB gene and a genetic modification in the fljBA genes.

19. The mutant of claim 1, containing a genetic modification in the guaB gene, a genetic modification in the fliC gene, and a genetic modification in the fljBA gene.

20. The mutant strain of claim 1, wherein the modification is selected from the group consisting of an insertion, a deletion, and a substitution of one or more nucleotides in said genes.

21. A vaccine for immunizing a veterinary species against a bacterial infection, said vaccine comprising: a pharmaceutically effective or an immunizing amount of a mutant strain according to claim 1; and a pharmaceutically acceptable carrier or diluent.

22. The vaccine of claim 21, wherein said mutant strain encodes and expresses a foreign antigen.

23. The vaccine of claim 21, wherein said mutant strain comprises a plasmid, which encodes and expresses an exogenous gene in a eukaryotic cell.

24. A method of immunizing a veterinary species against a bacterial infection, said method comprising the step of: administering to a veterinary species in need thereof an immunizing amount of a mutant strain according to claim 1 and/or a vaccine according to claim 21.

25. The method of claim 24 wherein the veterinary species is poultry.

26. The method of claim 24 wherein the mutant strain is an attenuated strain of S. enterica or E. coli.

27. The method of claim 24, wherein the mutant strain and/or the vaccine is administered via oral, nasal or parenteral routes.

28. (canceled)

29. A method for a serological distinction between vaccinated animals and animals infected by a wild-type strain, wherein the vaccinated animals have been immunized with a mutant bacterial strain comprising an inactivated a flagellin gene, comprising the steps of:
assaying animals for the presence of antibodies raised against flagellin; and
distinguishing infected animals from vaccinated animals based on the presence or absence of said antibodies.

30. The method of claim 29, wherein said antibodies are generated by an animal infected with a wild-type strain, but not by an animal that has been vaccinated with a mutant strain according to claim 1 in which a flagellin gene is has been inactivated.

31. The method of claim 29, wherein the presence of said antibodies indicates the presence of wild-type strains and infection.

32. The method of claim 29, wherein animals infected by Salmonellae are distinguished from animals that have been immunized with an attenuated live vaccine according to claim 21.

33. The method of claim 29, wherein the animals are assayed for antibodies raised against FliC.

34. The method of claim 29, wherein the animal is a veterinary species.

35. The method of claim 34, wherein the veterinary species is poultry.

36. The method of claim 35, wherein said poultry is a chicken.
